(19) United States
(12) Patent Application Publication
(10) Pub. No.: US 2024/0041853 A1
(43) Pub. Date: Feb. 8, 2024

(54) DOSAGE REGIMEN FOR A NR2B-NMDA RECEPTOR NAM FOR THE TREATMENT OF DEPRESSION

(71) Applicant: Novartis AG, Basel (CH)

(72) Inventors: Jang-Ho CHA, Cambridge, MA (US); Thomas FALLER, Basel (CH); Baltazar GOMEZ-MANCILLA, Rosenau (FR); Dimitris PAPANICOLAOU, East Hanover, NJ (US); Carman-Gabriela SERBAN, Basel (CH); Florian VON RAISON, Rueil-Malmaison (FR)

(21) Appl. No.: 18/255,423

(22) PCT Filed: Dec. 3, 2021
(86) PCT No.: PCT/IB2021/061268

§ 371 Date: Jun. 1, 2023

Related U.S. Application Data
(60) Provisional application No. 63/121,508, filed on Dec. 4, 2020.
(60) Provisional application No. 63/121,513, filed on Dec. 4, 2020.

(51) Int. Cl. A61K 31/4439; A61K 45/06; A61P 25/24

(52) U.S. Cl. CPC A61K 31/4439; A61K 45/06; A61P 25/24

(57) ABSTRACT

The invention relates to a dosage regimen of Compound (I), or a pharmaceutically acceptable salt thereof, for the treatment of diseases or disorders mediated by negative allosteric modulation or inhibition of NR2B-NMDA receptor including, but not limited to, major depressive disorder, treatment resistant depression and suicidality. The invention also relates to the use of Compound (I), or a pharmaceutically acceptable salt thereof, in the treatment of major depressive disorder in patients with suicidal ideation with intent.

### FIELD OF THE INVENTION

The invention relates to a dosage regimen of Compound (I), or a pharmaceutically acceptable salt thereof, for the treatment of diseases or disorders mediated by negative allosteric modulation or inhibition of NR2B-NMDA receptor including, but not limited to, major depressive disorder, treatment resistant depression and suicidality. The invention also relates to the use of Compound (I), or a pharmaceutically acceptable salt thereof, in the treatment of major depressive disorder in patients with suicidal ideation with intent.

### BACKGROUND OF THE INVENTION

Depression is a serious and life-threatening condition with high rates of morbidity and a chronic disease course. It is a common illness worldwide, with more than 264 million people affected (WHO 2020). Prevalence rates vary by age, peaking in older adulthood (above 7.5% among females aged 55-74 years, and above 5.5% among males (WHO 2017). When long-lasting and with moderate or severe intensity, depression may become a serious health condition. It can cause the affected person to suffer greatly, unable to work, maintain relationships, attend to self-care, and in the most severe cases patients may become hospitalized or attempt or commit suicide.

Major depressive disorder (MDD) is the psychiatric diagnosis most commonly associated with suicide. Close to 800,000 people die due to suicide every year. Suicide is the second leading cause of death in 15-29 year-olds (WHO 2020). Suicidal ideation is prevalent and appears to be a precondition for suicide attempts among psychiatric patients with MDD (McAuliffe 2002; Sokero et al 2003; Coryell and Young 2005.) The time between the onset of suicidal ideation and suicide attempt is often very short and can be minutes or a few days (Deisenhammer et al 2009; Otsuka et al 2015), highlighting the need for urgent intervention and development of novel antidepressant therapies with a rapid onset.

Concerted efforts over the past 40 years have led to the introduction of safer, better tolerated, and easier-to-prescribe antidepressants, most notably selective serotonin reuptake inhibitors (SSRIs) and serotonin norepinephrine reuptake inhibitors (SNRIs). Nevertheless, about 30 to 40% of patients with MDD fail to respond to first-line treatments including oral antidepressant medications of all classes (SSRIs, SNRIs, tricyclic antidepressants (TCAs, etc.) and psychotherapy (Rush et al 2006). In addition, the onset to treatment response, even when effective, often takes at least four weeks, leading to greater suffering, expenses, and suicidal risk. Consideration of electroconvulsive therapy (ECT) for acute treatment of severe depression when a rapid response is required, or when other treatments have failed is recommended, however ECT use is limited by significant adverse reactions and additional interventional concerns, such as use of general anesthesia, seizure induction, cognitive deficits and memory loss. Therefore, there remains an ongoing high need for rapid acting, either more effective or better tolerated treatments that can in an effective way interrupt a depressive episode, reduce suicidality, and also able to prevent future depressive episodes.

Ketamine, which is an N-Methyl-D-Aspartate (NMDA) receptor antagonist, has been shown to be effective in MDD in off-label research. A clinical study completed by Berman et al 2000 was the first double-blind placebo-controlled crossover trial to demonstrate rapid antidepressant effects of ketamine following a single dose (0.5 mg/kg infused over 40 minutes) in 7 patients. After this initial study, additional trials showed a similar effect in patients with unipolar and bipolar depression, including treatment-resistance depression (TRD) (Zarate et al 2006; Zarate et al 2012; Diazgranados et al 2010; Lapidus et al 2014; Murrough et al 2013; Murrough et al 2013). Ketamine has also been shown to reduce suicidality (Katalinic et al 2013; Murrough et al 2015). SPRAVATO® (esketamine), a non-competitive NMDA receptor antagonist, is the first approved drug of this class for the treatment of TRD, and for the treatment of treatment of depressive symptoms in adults with major depressive disorder (MDD) with acute suicidal ideation or behavior. While both ketamine and SPRAVATO® have demonstrated certain level of efficacy and showed rapid mode of action, their safety profile is not without adverse events meaningful both for patients and clinicians and neither drug has been approved for the treatment of MDD in patients having suicidal ideation with intent. In fact, prescribing information for SPRAVATO® includes a black box warning of increased risk of suicidal thoughts and behaviors in pediatric and young adult patients taking antidepressants. Thus, there remains a need for a drug having a rapid mode of action, but with reduced side effects, for the treatment of not only depressive symptoms of MDD, but also the treatment/reduction of suicidality patents with MDD.

### SUMMARY OF THE INVENTION

The invention relates to a dosage regimen of Compound (I), or a pharmaceutically acceptable salt thereof, for the treatment of diseases or disorders mediated by negative allosteric modulation or inhibition of NR2B-NMDA receptor including, but not limited to, major depressive disorder, treatment resistant depression and suicidality. The invention also relates to the use of Compound (I), or a pharmaceutically acceptable salt thereof, in the treatment of major depressive disorder in patients with suicidal ideation with intent.

### DETAILED DESCRIPTION OF THE INVENTION

Compound (I), as used herein, is 6-((1S)-24(3aR,5R,6aS)-5-(2-fluorophenoxy)hexahydrocyclopenta[c]pyrrol-2(1H)-yl)-1-hydroxyethyl)pyridin-3-ol, of the following formula:

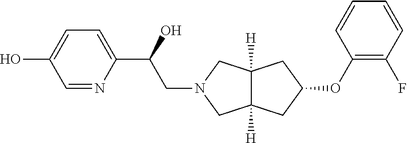 [SMILES: Oc1ccc([C@@H](O)CN2C[C@H]3C[C@H](Oc4ccccc4F)C[C@H]3C2)nc1]

is a NR2B-NMDA receptor non-allosteric modulator (NAM) which can be prepared as described in WO2016/049165, incorporated herein by reference.

Evidence suggests that the NR2B negative allosteric modulators (NAMs) MK-0657 (also known as CERC-301) and CP-101,606 have low frequencies of dissociative adverse events (Garner et al 2015; Pagnozzi et al 1995; Preskorn et al 2008). Although, the relative contribution of each individual subtype of NMDARs to the adverse effects of pan-NMDAR inhibition is poorly understood due to the lack of selective inhibitors for the various subtypes, taken together, this suggests that achieving safe, yet rapid-onset antidepressant efficacy is feasible with a compound selectively inhibiting NR2B receptor.

Compound (I), or a pharmaceutically acceptable salt thereof, is a highly potent, selective and reversible low molecular weight NR2B-NMDA receptor NAM. Compound (I) is for the rapid reduction of depressive symptoms in patients with MDD, including treatment resistant depression and suicidality. This treatment is intended to allow patients to rapidly achieve a significant improvement of their depressive symptoms, and suicidality. Moreover, patients having MDD with suicidality, often require 4-5 days of hospitalization. Compound (I), having rapid-onset of efficacy, can reduce the number of days a patient is hospitalized and therefore provide a benefit over other antidepressants that take at least 4 weeks for a patient to respond to treatment.

Compound (I), or a pharmaceutically acceptable salt thereof, is intended to treat suicidality, the symptoms of suicidality, including but not limited to, suicidal-ideation, suicidal-behavior and self-harm, alone or in conjunction with mental illness, including but not limited to, major depressive disorder. In particular, Compound (I), or a pharmaceutically acceptable salt thereof, is intended for the treatment of major depressive disorder in patients with suicidal ideation with intent.

As used herein, the terms “salt”, “salts” or “salt form” refers to an acid addition or base addition salt of a respective compound, e.g. the compounds specified herein (e.g. Compound (I) or further pharmaceutical active ingredient, for example, as defined herein). “Salts” include in particular “pharmaceutically acceptable salts”. The term “pharmaceutically acceptable salts” refers to salts that retain the biological effectiveness and properties of the compounds and, which typically are not biologically or otherwise undesirable. The compounds, as specified herein (e.g. Compound (I) or further pharmaceutical active ingredient, for example, as defined herein), may be capable of forming acid and/or base salts by virtue of the presence of amino and/or carboxyl groups or groups similar thereto. The compound of the invention is capable of forming acid addition salts, thus, as used herein, the term pharmaceutically acceptable salt of Compound (I) means a pharmaceutically acceptable acid addition salt of Compound (I).

Pharmaceutically acceptable acid addition salts can be formed with inorganic acids and organic acids.

Inorganic acids from which salts can be derived include, for example, hydrochloric acid, hydrobromic acid, sulfuric acid, nitric acid, phosphoric acid, and the like.

Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like.

Pharmaceutically acceptable base addition salts can be formed with inorganic and organic bases.

Inorganic bases from which salts can be derived include, for example, ammonium salts and metals from columns I to XII of the periodic table. In certain embodiments, the salts are derived from sodium, potassium, ammonium, calcium, magnesium, iron, silver, zinc, and copper; particularly suitable salts include ammonium, potassium, sodium, calcium and magnesium salts.

Organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine and tromethamine.

Pharmaceutically acceptable salts can be synthesized from a basic or acidic moiety, by conventional chemical methods. Generally, such salts can be prepared by reacting the free acid forms of the compound with a stoichiometric amount of the appropriate base (such as Na, Ca, Mg, or K hydroxide, carbonate, bicarbonate or the like), or by reacting the free base form of the compound with a stoichiometric amount of the appropriate acid. Such reactions are typically carried out in water or in an organic solvent, or in a mixture of the two. Generally, use of non-aqueous media like ether, ethyl acetate, ethanol, isopropanol, or acetonitrile is desirable, where practicable. Lists of additional suitable salts can be found, e.g., in “Remington's Pharmaceutical Sciences”, 22^{nd} edition, Mack Publishing Company (2013); and in “Handbook of Pharmaceutical Salts: Properties, Selection, and Use” by Stahl and Wermuth (Wiley-VCH, Weinheim, 2011, 2^{nd} edition).

Compound (I) or a pharmaceutically acceptable salt thereof can be administered by conventional route, for example orally, sublingually, or intravenously. Intravenous administration is in the form of solutions, nano-suspensions and lipid based formulations as a bolus, slow injection, infusion or drip, which can be manufactured according to pharmaceutical techniques as known in the art (for example in “Remington Essentials of Pharmaceutics, 2013, 1^{st} Edition, edited by Linda Felton, published by Pharmaceutical Press 2012, ISBN 978 0 85711 105 0; in particular Chapter 30), wherein pharmaceutical excipients are, for example, as described in “Handbook of Pharmaceutical Excipients, 2012, 7^{th} Edition, edited by Raymond C. Rowe, Paul J. Sheskey, Walter G. Cook and Marian E. Fenton, ISBN 978 0 85711 027 5”.

Compound (I), or a pharmaceutically acceptable salt thereof, is administered intravenously in an amount from about 0.0048 mg/kg to about 0.32 mg/kg, in particular 0.0048 mg/kg, 0.016 mg/kg, mg/kg, 0.16 mg/kg and 0.32 mg/kg of patient body weight. Compound (I), or a pharmaceutically acceptable salt thereof can be administered twice a week, once a week, once every other week, once a month or once every six weeks. Compound (I) can also be administered orally, sub-lingual, buccal, intravenously, or intranasally. When administered via intravenous infusion, the infusion time is between 20 to 90 minutes, in particular 40 minutes. In particular, Compound (I) is administered as a free base in the doses listed above.

Compound (I), or a pharmaceutically acceptable salt thereof, can also be administered as a single dose, for example 0.16 mg/kg and 0.048 mg/kg, given once to treat patients with major depressive disorder who have suicidal ideation with intent.

In one embodiment, Compound (I), or a pharmaceutically acceptable salt thereof, is administered in an amount of about 0.32 mg/kg twice a week, once a week, once every other week, once a month or once every six weeks. In another embodiment Compound (I), or a pharmaceutically acceptable salt thereof, is administered in an amount of about 0.32 mg/kg once every other week or once a month.

In another embodiment, Compound (I), or a pharmaceutically acceptable salt thereof, is administered in an amount of about 0.16 mg/kg twice a week, once a week, once every other week, once a month or once every six weeks. In another embodiment Compound (I), or a pharmaceutically acceptable salt thereof, is administered in an amount of about 0.16 mg/kg once every other week or once a month.

In another embodiment, Compound (I) or a pharmaceutically acceptable salt thereof is administered in an amount of about 0.048 mg/kg twice a week, once a week, once every other week, once a month or once every six weeks. In another embodiment Compound (I), or a pharmaceutically acceptable salt thereof, is administered in an amount of about 0.048 mg/kg once a week or once every other week.

In another embodiment, Compound (I) or a pharmaceutically acceptable salt thereof is administered in an amount of about 0.016 mg/kg twice a week, once a week, once every other week, once a month or once every six weeks.

In another embodiment, Compound (I) or a pharmaceutically acceptable salt thereof is administered in an amount of about 0.0048 mg/kg twice a week, once a week, once every other week, once a month or once every six weeks.

It has been found, in a proof of concept study carried out in patients having treatment resistant depression, that a dose of 0.16 mg/kg of Compound (I) administered once every week or once every other week is as effective as a dose of 0.32 mg/kg administered once a week or once every other week. This efficacy occurred within 24 hours, and was maintained at 6 weeks after repeated dosing.

Compound (I), or a pharmaceutically acceptable salt thereof, can be administered in conjunction with another antidepressant. Examples of antidepressants include, but are not limited to, selective serotonin reuptake inhibitors (SSRIs), such as fluoxetine (Prozac®), paroxetine (Paxil®, Pexeva®), sertraline (Zoloft®), citalopram (Celexa®) and escitalopram (Lexapro®); serotonin and norepinephrine reuptake inhibitors (SSRIs), such as duloxetine (Cymbalta®), venlafaxine (Effexor XR®), desvenlafaxine (Pristiq®) and levomilnacipran (Fetzima®); atypical antidepressants, such as trazodone, mirtazapine (Remeron®), vortioxetine (Trintellix®), vilazodone (Viibryd®) and bupropion (Wellbutrin SR, Wellbutrin XL, others); tricyclic antidepressants, such as imipramine (Tofranil), nortriptyline (Pamelor®), amitriptyline, doxepin and desipramine (Norpramin)®; Monoamine oxidase inhibitors (MAOIs), such as tranylcypromine (Parnate®), phenelzine (Nardil®) and isocarboxazid (Marplan®).

Compound (I), or a pharmaceutically acceptable salt thereof, can be administered in conjunction with another antidepressant (as listed above) and an antipsychotic or mood stabilizer. Examples of antipsychotics or mood stabilizers include, but are not limited to, lithium carbonate, carbamazepine (Tegretol®), valproic acid (Depakote®, Depakene®), gabpentin (Neurontin®), topiramate (Topamax®), lamotrigine (Lomictal®), olanzapine (Zyprexa®), clozapine (Clozaril®), and risperidone (Risperdal®).

### EMBODIMENTS

### Embodiments (a)

1a. Compound (I), or a pharmaceutically acceptable salt thereof, for use in the treatment of major depressive disorder comprising administration of Compound (I), or a pharmaceutically acceptable salt thereof, in an amount of from about 0.0048 mg/kg to about 0.32 mg/kg, in particular 0.0048 mg/kg, 0.016 mg/kg, 0.048 mg/kg, 0.16 mg/kg and 0.32 mg/kg of patient body weight.
2a. Compound (I), or a pharmaceutically acceptable salt thereof, for use in the treatment of treatment resistant depression comprising administration of Compound (I), or a pharmaceutically acceptable salt thereof, in an amount of from about 0.0048 mg/kg to about mg/kg, in particular 0.0048 mg/kg, 0.016 mg/kg, 0.048 mg/kg, 0.16 mg/kg and 0.32 mg/kg of patient body weight.
3a. Compound (I), or a pharmaceutically acceptable salt thereof, for use in the treatment of depressive symptoms comprising administration of Compound (I), or a pharmaceutically acceptable salt thereof, in an amount of from about 0.0048 mg/kg to about 0.32 mg/kg, in particular 0.0048 mg/kg, 0.016 mg/kg, 0.048 mg/kg, 0.16 mg/kg and 0.32 mg/kg of patient body weight.
4a. Compound (I), or a pharmaceutically acceptable salt thereof, for use in the treatment of suicidality in major depressive disorder comprising administration of Compound (I), or a pharmaceutically acceptable salt thereof, in an amount of from about 0.0048 mg/kg to about mg/kg, in particular 0.0048 mg/kg, 0.016 mg/kg, 0.048 mg/kg, 0.16 mg/kg and 0.32 mg/kg of patient body weight.
5a. Compound (I), or a pharmaceutically acceptable salt thereof, for use in the treatment of major depressive disorder with suicidal ideation comprising administration of Compound (I), or a pharmaceutically acceptable salt thereof, in an amount of from about 0.0048 mg/kg to about 0.32 mg/kg, in particular 0.0048 mg/kg, 0.016 mg/kg, 0.048 mg/kg, 0.16 mg/kg and 0.32 mg/kg of patient body weight.
6a. Compound (I), or a pharmaceutically acceptable salt thereof, for use in the treatment of major depressive disorder with suicidal ideation and suicidal intent comprising administration of Compound (I), or a pharmaceutically acceptable salt thereof, in an amount of from about 0.0048 mg/kg to about 0.32 mg/kg, in particular 0.0048 mg/kg, 0.016 mg/kg, 0.048 mg/kg, 0.16 mg/kg and 0.32 mg/kg of patient body weight.
7a. Compound (I), or a pharmaceutically acceptable salt thereof, for use in the treatment of major depressive disorder with suicidal behavior comprising administration of Compound (I), or a pharmaceutically acceptable salt thereof, in an amount of from about 0.0048 mg/kg to about 0.32 mg/kg, in particular 0.0048 mg/kg, 0.016 mg/kg, 0.048 mg/kg, 0.16 mg/kg and 0.32 mg/kg of patient body weight.
8a. Compound (I), or a pharmaceutically acceptable salt thereof, for use in the treatment of self-harm in major depressive disorder comprising administration of Compound (I), or a pharmaceutically acceptable salt thereof, in an amount of from about 0.0048 mg/kg to about mg/kg, in particular 0.0048 mg/kg, 0.016 mg/kg, 0.048 mg/kg, 0.16 mg/kg and 0.32 mg/kg of patient body weight.
9a. Compound (I), or a pharmaceutically acceptable salt thereof, for use according to any one of embodiments 1a to 8a wherein the use is in conjunction with one or more other antidepressants.
10a. Compound (I), or a pharmaceutically acceptable salt thereof, for use according to any one of embodiments 1a to 8a wherein the use is in conjunction with psychotherapy.
11a. Compound (I), or a pharmaceutically acceptable salt thereof, for use according to any one of embodiments 1a to 8a wherein the use is in conjunction with another antidepressant and psychotherapy.
12a. Compound (I), or a pharmaceutically acceptable salt thereof, for use according to any one of embodiments 1a to 8a wherein Compound (I), or a pharmaceutically acceptable salt thereof is administered intravenously.
13a. Compound (I), or a pharmaceutically acceptable salt thereof, for use according to any one of embodiments 1a to 8a wherein Compound (I), or a pharmaceutically acceptable salt thereof is administered intravenously through infusion over a timeframe of about 40 minutes.
14a. Compound (I), or a pharmaceutically acceptable salt thereof, for use according to any one of embodiments 1a to 8a wherein Compound (I), or a pharmaceutically acceptable salt thereof is administered twice a week, once a week, once every other week, once a month or once every six weeks.
15a. Compound (I), or a pharmaceutically acceptable salt thereof, for use according to embodiment 14a wherein Compound (I) or a pharmaceutically acceptable salt thereof is administered in an amount of about 0.16 mg/kg once every other week or once a month.
16a. Compound (I), or a pharmaceutically acceptable salt thereof, for use according to embodiment 14a wherein Compound (I) or a pharmaceutically acceptable salt thereof is administered in an amount of about 0.048 mg/kg once a week or once every other week.
17a. Compound (I), or a pharmaceutically acceptable salt thereof, for use according to any one of embodiments 1a to 8a wherein the use is in conjunction with one or more other antidepressants and an antipsychotic or mood stabilizer.

### Embodiments (b)

1b. A pharmaceutical composition comprising Compound (I), or a pharmaceutically acceptable salt thereof, for use in the treatment of major depressive disorder comprising administration of Compound (I), or a pharmaceutically acceptable salt thereof, in an amount of about 0.0048 mg/kg to about 0.32 mg/kg, in particular 0.0048 mg/kg, 0.016 mg/kg, 0.048 mg/kg, 0.16 mg/kg and 0.32 mg/kg of patient body weight.
2b. A pharmaceutical composition comprising Compound (I), or a pharmaceutically acceptable salt thereof, for use in the treatment of treatment resistant depression comprising administration of Compound (I), or a pharmaceutically acceptable salt thereof, in an amount of from about 0.0048 mg/kg to about 0.32 mg/kg, in particular 0.0048 mg/kg, 0.016 mg/kg, 0.048 mg/kg, 0.16 mg/kg and 0.32 mg/kg of patient body weight.
3b. A pharmaceutical composition comprising Compound (I), or a pharmaceutically acceptable salt thereof, for use in the treatment of depressive symptoms comprising administration of Compound (I), or a pharmaceutically acceptable salt thereof, in an amount of from about mg/kg to about 0.32 mg/kg, in particular 0.0048 mg/kg, 0.016 mg/kg, 0.048 mg/kg, 0.16 mg/kg and 0.32 mg/kg of patient body weight.
4b. A pharmaceutical composition comprising Compound (I), or a pharmaceutically acceptable salt thereof, for use in the treatment suicidality in major depressive disorder comprising administration of Compound (I), or a pharmaceutically acceptable salt thereof, in an amount of from about 0.0048 mg/kg to about 0.32 mg/kg, in particular 0.0048 mg/kg, 0.016 mg/kg, 0.048 mg/kg, 0.16 mg/kg and 0.32 mg/kg of patient body weight.
5b. A pharmaceutical composition comprising Compound (I), or a pharmaceutically acceptable salt thereof, for use in the treatment of major depressive disorder with suicidal ideation comprising administration of Compound (I), or a pharmaceutically acceptable salt thereof, in an amount of from about 0.0048 mg/kg to about 0.32 mg/kg, in particular 0.0048 mg/kg, 0.016 mg/kg, 0.048 mg/kg, 0.16 mg/kg and 0.32 mg/kg of patient body weight.
6b. A pharmaceutical composition comprising Compound (I), or a pharmaceutically acceptable salt thereof, for use in the treatment of major depressive disorder with suicidal ideation and suicidal intent comprising administration of Compound (I), or a pharmaceutically acceptable salt thereof, in an amount of from about 0.0048 mg/kg to about 0.32 mg/kg, in particular 0.0048 mg/kg, 0.016 mg/kg, 0.048 mg/kg, 0.16 mg/kg and 0.32 mg/kg of patient body weight.
7b. A pharmaceutical composition comprising Compound (I), or a pharmaceutically acceptable salt thereof, for use in the treatment of major depressive disorder with suicidal behavior comprising administration of Compound (I), or a pharmaceutically acceptable salt thereof, in an amount of from about 0.0048 mg/kg to about 0.32 mg/kg, in particular 0.0048 mg/kg, 0.016 mg/kg, 0.048 mg/kg, 0.16 mg/kg and 0.32 mg/kg of patient body weight.
8b. A pharmaceutical composition comprising Compound (I), or a pharmaceutically acceptable salt thereof, for use in the treatment of self-harm in major depressive disorder comprising administration of Compound (I), or a pharmaceutically acceptable salt thereof, in an amount of from about 0.0048 mg/kg to about 0.32 mg/kg, in particular 0.0048 mg/kg, 0.016 mg/kg, 0.048 mg/kg, 0.16 mg/kg and 0.32 mg/kg of patient body weight.
9b. A pharmaceutical composition comprising Compound (I), or a pharmaceutically acceptable salt thereof, for use according to any one of embodiments 1 b to 8b wherein the use is in conjunction with one or more other antidepressants.
10b. A pharmaceutical composition comprising Compound (I), or a pharmaceutically acceptable salt thereof, for use according to any one of embodiments 1b to 8b wherein the use is in conjunction with psychotherapy.
11b. A pharmaceutical composition comprising Compound (I), or a pharmaceutically acceptable salt thereof, for use according to any one of embodiments 1b to 8b wherein the use is in conjunction with another antidepressant and psychotherapy.
12b. A pharmaceutical composition comprising Compound (I), or a pharmaceutically acceptable salt thereof, for use according to any one of embodiments 1b to 8b wherein the pharmaceutical composition is administered intravenously.
13b. A pharmaceutical composition comprising Compound (I), or a pharmaceutically acceptable salt thereof, for use according to any one of embodiments 1b to 8b wherein the pharmaceutical composition is administered intravenously through infusion over a timeframe of about 40 minutes.
14b. A pharmaceutical composition comprising Compound (I), or a pharmaceutically acceptable salt thereof, for use according to any one of embodiments 1 b to 8b wherein the pharmaceutical composition is administered twice a week, once a week, once every other week, once a month or once every six weeks.
15b. A pharmaceutical composition comprising Compound (I), or a pharmaceutically acceptable salt thereof, for use according to embodiment 14b wherein the pharmaceutical composition is administered in an amount of about 0.16 mg/kg once every other week or once a month.
16b. A pharmaceutical composition comprising Compound (I), or a pharmaceutically acceptable salt thereof, for use according to embodiment 14b wherein the pharmaceutical composition is administered in an amount of about 0.048 mg/kg once a week or once every other week.
17b. A pharmaceutical composition comprising Compound (I), or a pharmaceutically acceptable salt thereof, for use according to any one of embodiments 1b to 8b wherein the use is in conjunction with one or more other antidepressants and an antipsychotic or mood stabilizer.

### Embodiments (c)

1c. Use of Compound (I), or a pharmaceutically acceptable salt thereof, for the manufacture of a medicament for the treatment of major depressive disorder wherein Compound (I), or a pharmaceutically acceptable salt thereof, is administered in an amount of from about 0.0048 mg/kg to about 0.32 mg/kg, in particular 0.0048 mg/kg, 0.016 mg/kg, 0.048 mg/kg, 0.16 mg/kg and 0.32 mg/kg of patient body weight.
2c. Use of Compound (I), or a pharmaceutically acceptable salt thereof, for the manufacture of a medicament for the treatment of treatment resistant depression wherein Compound (I), or a pharmaceutically acceptable salt thereof, is administered in an amount of from about 0.0048 mg/kg to about 0.32 mg/kg, in particular 0.0048 mg/kg, 0.016 mg/kg, 0.048 mg/kg, 0.16 mg/kg and 0.32 mg/kg of patient body weight.
3c. Use of Compound (I), or a pharmaceutically acceptable salt thereof, for the manufacture of a medicament for the treatment of depressive symptoms wherein Compound (I), or a pharmaceutically acceptable salt thereof, is administered in an amount of from about 0.0048 mg/kg to about 0.32 mg/kg, in particular 0.0048 mg/kg, 0.016 mg/kg, 0.048 mg/kg, 0.16 mg/kg and 0.32 mg/kg of patient body weight.
4c. Use of Compound (I), or a pharmaceutically acceptable salt thereof, for the manufacture of a medicament for the treatment of suicidality in a patient having major depressive disorder wherein Compound (I), or a pharmaceutically acceptable salt thereof is administered in an amount of from about 0.0048 mg/kg to about 0.32 mg/kg, in particular 0.0048 mg/kg, 0.016 mg/kg, 0.048 mg/kg, 0.16 mg/kg and 0.32 mg/kg of patient body weight.
5c. Use of Compound (I), or a pharmaceutically acceptable salt thereof, for the manufacture of a medicament for the treatment of a patient having major depressive disorder with suicidal ideation wherein Compound (I), or a pharmaceutically acceptable salt thereof is administered in an amount of from about 0.0048 mg/kg to about 0.32 mg/kg, in particular 0.0048 mg/kg, 0.016 mg/kg, 0.048 mg/kg, 0.16 mg/kg and 0.32 mg/kg of patient body weight.
6c. Use of Compound (I), or a pharmaceutically acceptable salt thereof, for the manufacture of a medicament for the treatment of a patient having major depressive disorder with suicidal ideation and suicidal intent wherein Compound (I), or a pharmaceutically acceptable salt thereof, is administered in an amount of from about 0.0048 mg/kg to about 0.32 mg/kg, in particular 0.0048 mg/kg, 0.016 mg/kg, 0.048 mg/kg, 0.16 mg/kg and 0.32 mg/kg of patient body weight.
7c. Use of Compound (I), or a pharmaceutically acceptable salt thereof, for the manufacture of a medicament for the treatment of a patient having major depressive disorder with suicidal behavior wherein Compound (I), or a pharmaceutically acceptable salt thereof, is administered in an amount of from about 0.0048 mg/kg to about 0.32 mg/kg, in particular 0.0048 mg/kg, 0.016 mg/kg, 0.048 mg/kg, 0.16 mg/kg and 0.32 mg/kg of patient body weight.
8c. Use of Compound (I), or a pharmaceutically acceptable salt thereof, for the manufacture of a medicament for the treatment of self-harm in a patient having major depressive disorder wherein Compound (I), or a pharmaceutically acceptable salt thereof is administered in an amount of from about 0.0048 mg/kg to about 0.32 mg/kg, in particular 0.0048 mg/kg, 0.016 mg/kg, 0.048 mg/kg, 0.16 mg/kg and 0.32 mg/kg of patient body weight.
9c. Use of Compound (I), or a pharmaceutically acceptable salt thereof, according to any one of embodiments 1c to 8c wherein the use is in conjunction with one or more antidepressants.
10c. Use of Compound (I), or a pharmaceutically acceptable salt thereof, according to any one of embodiments 1c to 8c wherein the use is in conjunction with psychotherapy.
11c. Use of Compound (I), or a pharmaceutically acceptable salt thereof, according to any one of embodiments 1c to 8c wherein the use is in conjunction with antidepressant and psychotherapy.
12c. Use of Compound (I), or a pharmaceutically acceptable salt thereof, according to any one of embodiments 1c to 8c wherein Compound (I), or a pharmaceutically acceptable salt thereof is administered intravenously.
13c. Use of Compound (I), or a pharmaceutically acceptable salt thereof, according to any one of embodiments 1c to 8c wherein Compound (I), or a pharmaceutically acceptable salt thereof is administered intravenously through infusion over a timeframe of about 40 minutes.
14c. Use of Compound (I), or a pharmaceutically acceptable salt thereof, according to any one of embodiments 1c to 8c wherein the pharmaceutical composition is administered twice a week, once a week, once every other week, once a month or once every six weeks.
15c. Use of Compound (I), or a pharmaceutically acceptable salt thereof, according to embodiment 14c wherein the pharmaceutical composition is administered in an amount of about 0.16 mg/kg once every other week or once a month.
16c. Use of Compound (I), or a pharmaceutically acceptable salt thereof, according to embodiment 14c wherein the pharmaceutical composition is administered in an amount of about 0.048 mg/kg once a week or once every other week.
17c. Use of Compound (I), or a pharmaceutically acceptable salt thereof, according to any one of embodiments 1c to 8c wherein the use is in conjunction with one or more antidepressants and an antipsychotic or mood stabilizer.

### Embodiments (d)

1d. Use of a pharmaceutical composition comprising Compound (I), or a pharmaceutically acceptable salt thereof, for the manufacture of a medicament for the treatment of major depressive disorder wherein Compound (I), or a pharmaceutically acceptable salt thereof, is administered in an amount of from about 0.0048 mg/kg to about 0.32 mg/kg, in particular mg/kg, 0.016 mg/kg, 0.048 mg/kg, 0.16 mg/kg and 0.32 mg/kg of patient body weight.
2d. Use of a pharmaceutical composition comprising Compound (I), or a pharmaceutically acceptable salt thereof, for the manufacture of a medicament for the treatment of treatment resistant depression wherein Compound (I), or a pharmaceutically acceptable salt thereof, is administered in an amount of from about 0.0048 mg/kg to about 0.32 mg/kg, in particular mg/kg, 0.016 mg/kg, 0.048 mg/kg, 0.16 mg/kg and 0.32 mg/kg of patient body weight.
3d. Use of a pharmaceutical composition comprising Compound (I), or a pharmaceutically acceptable salt thereof, for the manufacture of a medicament for the treatment of depressive symptoms wherein Compound (I), or a pharmaceutically acceptable salt thereof, is administered in an amount of from about 0.0048 mg/kg to about 0.32 mg/kg, in particular mg/kg, 0.016 mg/kg, 0.048 mg/kg, 0.16 mg/kg and 0.32 mg/kg of patient body weight.
4d. Use of a pharmaceutical composition comprising Compound (I), or a pharmaceutically acceptable salt thereof, for the manufacture of a medicament for the treatment of suicidality in a patient having major depressive disorder wherein Compound (I), or a pharmaceutically acceptable salt thereof is administered in an amount of from about 0.0048 mg/kg to about 0.32 mg/kg, in particular 0.0048 mg/kg, 0.016 mg/kg, 0.048 mg/kg, 0.16 mg/kg and 0.32 mg/kg of patient body weight.
5d. Use of a pharmaceutical composition comprising Compound (I), or a pharmaceutically acceptable salt thereof, for the manufacture of a medicament for the treatment of a patient having major depressive disorder with suicidal ideation wherein Compound (I), or a pharmaceutically acceptable salt thereof is administered in an amount of from about 0.0048 mg/kg to about 0.32 mg/kg, in particular 0.0048 mg/kg, 0.016 mg/kg, 0.048 mg/kg, 0.16 mg/kg and 0.32 mg/kg of patient body weight.
6d. Use of a pharmaceutical composition comprising Compound (I), or a pharmaceutically acceptable salt thereof, for the manufacture of a medicament for the treatment of a patient having major depressive disorder with suicidal ideation and suicidal intent wherein Compound (I), or a pharmaceutically acceptable salt thereof, is administered in an amount of from about 0.0048 mg/kg to about 0.32 mg/kg, in particular 0.0048 mg/kg, 0.016 mg/kg, 0.048 mg/kg, 0.16 mg/kg and 0.32 mg/kg of patient body weight.
7d. Use of a pharmaceutical composition comprising Compound (I), or a pharmaceutically acceptable salt thereof, for the manufacture of a medicament for the treatment of a patient having major depressive disorder with suicidal behavior wherein Compound (I), or a pharmaceutically acceptable salt thereof, is administered in an amount of from about 0.0048 mg/kg to about 0.32 mg/kg, in particular 0.0048 mg/kg, 0.016 mg/kg, 0.048 mg/kg, 0.16 mg/kg and 0.32 mg/kg of patient body weight.
8d. Use of a pharmaceutical composition comprising Compound (I), or a pharmaceutically acceptable salt thereof, for the manufacture of a medicament for the treatment of self-harm in a patient having major depressive disorder wherein Compound (I), or a pharmaceutically acceptable salt thereof is administered in an amount of from about 0.0048 mg/kg to about 0.32 mg/kg, in particular 0.0048 mg/kg, 0.016 mg/kg, 0.048 mg/kg, 0.16 mg/kg and 0.32 mg/kg of patient body weight.
9d. Use of a pharmaceutical composition comprising Compound (I), or a pharmaceutically acceptable salt thereof, according to any one of embodiments 1d to 8d wherein the use is in conjunction with one or more antidepressants.
10d. Use of Compound (I), or a pharmaceutically acceptable salt thereof, according to any one of embodiments 1d to 8d wherein the use is in conjunction with psychotherapy.
11d. Use of Compound (I), or a pharmaceutically acceptable salt thereof, according to any one of embodiments 1d to 8d wherein the use is in conjunction with antidepressant and psychotherapy.
12d. Use of Compound (I), or a pharmaceutically acceptable salt thereof, according to any one of embodiments 1d to 8d wherein Compound (I), or a pharmaceutically acceptable salt thereof is administered intravenously.
13d. Use of Compound (I), or a pharmaceutically acceptable salt thereof, according to any one of embodiments 1d to 8d wherein Compound (I), or a pharmaceutically acceptable salt thereof is administered intravenously through infusion over a timeframe of about 40 minutes.
14d. Use of Compound (I), or a pharmaceutically acceptable salt thereof, according to any one of embodiments 1 d to 8d wherein the pharmaceutical composition is administered twice a week, once a week, once every other week, once a month or once every six weeks.
15d. Use of Compound (I), or a pharmaceutically acceptable salt thereof, according to embodiment 14d wherein the pharmaceutical composition is administered in an amount of about 0.16 mg/kg once every other week or once a month.
16d. Use of Compound (I), or a pharmaceutically acceptable salt thereof, according to embodiment 14d wherein the pharmaceutical composition is administered in an amount of about mg/kg once a week or once every other week.
17d. Use of a pharmaceutical composition comprising Compound (I), or a pharmaceutically acceptable salt thereof, according to any one of embodiments 1d to 8d wherein the use is in conjunction with one or more antidepressants and an antipsychotic or mood stabilizer.

### Embodiments (e)

1e. A method for the treatment of major depressive disorder comprising administration of Compound (I), or a pharmaceutically acceptable salt thereof, to a patient in need thereof, in an amount of from about 0.0048 mg/kg to about 0.32 mg/kg, in particular 0.0048 mg/kg, 0.016 mg/kg, 0.048 mg/kg, 0.16 mg/kg and 0.32 mg/kg of patient body weight.
2e. A method for the treatment of treatment resistant depression comprising administration of Compound (I), or a pharmaceutically acceptable salt thereof, to a patient in need thereof, in an amount of from about 0.0048 mg/kg to about 0.32 mg/kg, in particular 0.0048 mg/kg, 0.016 mg/kg, 0.048 mg/kg, 0.16 mg/kg and 0.32 mg/kg of patient body weight.
3e. A method for the treatment of depressive symptoms comprising administration of Compound (I), or a pharmaceutically acceptable salt thereof, to a patient in need thereof, in an amount of from about 0.0048 mg/kg to about 0.32 mg/kg, in particular 0.0048 mg/kg, 0.016 mg/kg, 0.048 mg/kg, 0.16 mg/kg and 0.32 mg/kg of patient body weight.
4e. A method for the treatment of suicidality in a patient having major depressive disorder comprising administration of Compound (I), or a pharmaceutically acceptable salt thereof, to a patient in need thereof, in an amount of from about 0.0048 mg/kg to about 0.32 mg/kg, in particular 0.0048 mg/kg, 0.016 mg/kg, 0.048 mg/kg, 0.16 mg/kg and 0.32 mg/kg of patient body weight.
5e. A method for the treatment of major depressive disorder with suicidal ideation comprising administration of Compound (I), or a pharmaceutically acceptable salt thereof, to a patient in need thereof in an amount of from about 0.0048 mg/kg to about 0.32 mg/kg, in particular mg/kg, 0.016 mg/kg, 0.048 mg/kg, 0.16 mg/kg and 0.32 mg/kg of patient body weight.
6e. A method for the treatment of major depressive disorder with suicidal ideation and suicidal intent comprising administration of Compound (I), or a pharmaceutically acceptable salt thereof, to a patient in need thereof in an amount of from about 0.0048 mg/kg to about 0.32 mg/kg, in particular 0.0048 mg/kg, 0.016 mg/kg, 0.048 mg/kg, 0.16 mg/kg and 0.32 mg/kg of patient body weight.
7e. A method for the treatment major depressive disorder with suicidal behavior comprising administration of Compound (I), or a pharmaceutically acceptable salt thereof, in a patient in need thereof in an amount of from about 0.0048 mg/kg to about 0.32 mg/kg, in particular 0.0048 mg/kg, 0.016 mg/kg, 0.048 mg/kg, 0.16 mg/kg and 0.32 mg/kg of patient body weight.
8e. A method for the treatment of self-harm in major depressive disorder comprising administration of Compound (I), or a pharmaceutically acceptable salt thereof, to a patient in need thereof in an amount of from about 0.0048 mg/kg to about 0.32 mg/kg, in particular 0.0048 mg/kg, 0.016 mg/kg, 0.048 mg/kg, 0.16 mg/kg and 0.32 mg/kg of patient body weight.
9e. The method according to any one of embodiments 1e to 8e wherein treatment is in conjunction with one or more other antidepressants.
10e. The method according to any one of embodiments 1e to 8e wherein treatment is in conjunction with psychotherapy.
11e. The method according to any one of embodiments 1e to 8e wherein treatment is in conjunction with another antidepressant and psychotherapy.
12e. The method according to any one of embodiments 1e to 8e wherein Compound (I), or a pharmaceutically acceptable salt thereof is administered intravenously.
13e. The method according to any one of embodiments 1a to 8e wherein Compound (I), or a pharmaceutically acceptable salt thereof is administered intravenously through infusion over a timeframe of about 40 minutes.
14e. The method according to any one of embodiments 1a to 8e wherein Compound (I), or a pharmaceutically acceptable salt thereof is administered twice a week, once a week, once every other week, once a month or once every six weeks.
15e. The method according to embodiment 14e wherein Compound (I) or a pharmaceutically acceptable salt thereof is administered in an amount of about 0.16 mg/kg once every other week or once a month.
16e. The method according to embodiment 14e wherein Compound (I) or a pharmaceutically acceptable salt thereof is administered in an amount of about 0.048 mg/kg once a week or once every other week.
17e. The method according to any one of embodiments 1e to 8e wherein treatment is in conjunction with one or more other antidepressants and an antipsychotic or mood stabilizer.

### Embodiments (f)

1f. A method for the treatment of major depressive disorder comprising administration of a pharmaceutical composition comprising Compound (I), or a pharmaceutically acceptable salt thereof, to a patient in need thereof, in an amount of Compound (I), or a pharmaceutically acceptable salt thereof, from about 0.0048 mg/kg to about 0.32 mg/kg, in particular 0.0048 mg/kg, 0.016 mg/kg, 0.048 mg/kg, 0.16 mg/kg and 0.32 mg/kg of patient body weight.
2f. A method for the treatment of treatment resistant depression comprising administration of a pharmaceutical composition comprising Compound (I), or a pharmaceutically acceptable salt thereof, to a patient in need thereof, in an amount of Compound (I), or a pharmaceutically acceptable salt thereof, from about 0.0048 mg/kg to about 0.32 mg/kg, in particular 0.0048 mg/kg, 0.016 mg/kg, 0.048 mg/kg, 0.16 mg/kg and 0.32 mg/kg of patient body weight.
3f. A method for the treatment of depressive symptoms comprising administration of a pharmaceutical composition comprising Compound (I), or a pharmaceutically acceptable salt thereof, to a patient in need thereof, in an amount of Compound (I), or a pharmaceutically acceptable salt thereof, from about 0.0048 mg/kg to about 0.32 mg/kg, in particular 0.0048 mg/kg, 0.016 mg/kg, 0.048 mg/kg, 0.16 mg/kg and 0.32 mg/kg of patient body weight.
4f. A method for the treatment of suicidality in a patient having major depressive disorder comprising administration of a pharmaceutical composition comprising Compound (I), or a pharmaceutically acceptable salt thereof, to a patient in need thereof, in an amount of Compound (I), or a pharmaceutically acceptable salt thereof, from about 0.0048 mg/kg to about 0.32 mg/kg, in particular 0.0048 mg/kg, 0.016 mg/kg, 0.048 mg/kg, 0.16 mg/kg and 0.32 mg/kg of patient body weight.
5f. A method for the treatment of major depressive disorder with suicidal ideation comprising administration of a pharmaceutical composition comprising Compound (I), or a pharmaceutically acceptable salt thereof, to a patient in need thereof, in an amount of Compound (I), or a pharmaceutically acceptable salt thereof, from about 0.0048 mg/kg to about mg/kg, in particular 0.0048 mg/kg, 0.016 mg/kg, 0.048 mg/kg, 0.16 mg/kg and 0.32 mg/kg of patient body weight.
6f. A method for the treatment of major depressive disorder with suicidal ideation and suicidal intent comprising administration of a pharmaceutical composition comprising Compound (I), or a pharmaceutically acceptable salt thereof, to a patient in need thereof, in an amount of Compound (I), or a pharmaceutically acceptable salt thereof, from about 0.0048 mg/kg to about mg/kg, in particular 0.0048 mg/kg, 0.016 mg/kg, 0.048 mg/kg, 0.16 mg/kg and 0.32 mg/kg of patient body weight.
7f. A method for the treatment major depressive disorder with suicidal behavior comprising administration of a pharmaceutical composition comprising Compound (I), or a pharmaceutically acceptable salt thereof, in a patient in need thereof, in an amount of Compound (I), or a pharmaceutically acceptable salt thereof, from about 0.0048 mg/kg to about mg/kg, in particular 0.0048 mg/kg, 0.016 mg/kg, 0.048 mg/kg, 0.16 mg/kg and 0.32 mg/kg of patient body weight.
8f. A method for the treatment of self-harm in a patient having major depressive disorder comprising administration of a pharmaceutical composition comprising Compound (I), or a pharmaceutically acceptable salt thereof, to a patient in need thereof, in an amount of Compound (I), or a pharmaceutically acceptable salt thereof, from about 0.0048 mg/kg to about mg/kg, in particular 0.0048 mg/kg, 0.016 mg/kg, 0.048 mg/kg, 0.16 mg/kg and 0.32 mg/kg of patient body weight.
9f. The method according to any one of embodiments 1f to 8f wherein treatment is in conjunction with one or more other antidepressants.
10f. The method according to any one of embodiments 1f to 8f wherein treatment is in conjunction with psychotherapy.
11f. The method according to any one of embodiments 1f to 8f wherein treatment is in conjunction with another antidepressant and psychotherapy.
12f. The method according to any one of embodiments 1f to 8f wherein the pharmaceutical composition is administered intravenously.
13f. The method according to any one of embodiments 1f to 8f wherein the pharmaceutical composition is administered intravenously through infusion over a timeframe of about 40 minutes.
14f. The method according to any one of embodiments 1f to 8f wherein the pharmaceutical composition is administered twice a week, once a week, once every other week, once a month or once every six weeks.
15f. The method according to embodiment 13f wherein the pharmaceutical composition is administered in an amount of about 0.16 mg/kg once every other week or once a month.
16f. The method according to embodiment 13f wherein the pharmaceutical composition is administered in an amount of about 0.048 mg/kg once a week or once every other week.
17f. The method according to any one of embodiments 1f to 8f wherein treatment is in conjunction with one or more other antidepressants and an antipsychotic or mood stabilizer.

### Embodiments (g)

1g. Compound (I), or a pharmaceutically acceptable salt thereof, for use in the treatment suicidality in a patient having major depressive disorder.
2g. Compound (I), or a pharmaceutically acceptable salt thereof, for use in the treatment of a patient having major depressive disorder with suicidal ideation.
3g. Compound (I), or a pharmaceutically acceptable salt thereof, for use in the treatment of a patient having major depressive disorder with suicidal ideation and suicidal intent.
4g. Compound (I), or a pharmaceutically acceptable salt thereof, for use in the treatment of a patient having major depressive disorder with suicidal behavior.
5g. Compound (I), or a pharmaceutically acceptable salt thereof, for use in the treatment of self-harm in a patient having major depressive disorder.
6g. Compound (I), or a pharmaceutically acceptable salt thereof, for use according to any one of embodiments 1g to 5g wherein the use is in conjunction with one or more antidepressants.
7g. Compound (I), or a pharmaceutically acceptable salt thereof, for use according to any one of embodiments 1g to 5g wherein the use is in conjunction with psychotherapy.
8g. Compound (I), or a pharmaceutically acceptable salt thereof, for use according to any one of embodiments 1g to 5g wherein the use is in conjunction with one or more antidepressants and psychotherapy.
9g. Compound (I), or a pharmaceutically acceptable salt thereof, for use according to any one of embodiments 1g to 7g wherein Compound (I), or a pharmaceutically acceptable salt thereof, is administered intravenously.
10g. Compound (I), or a pharmaceutically acceptable salt thereof, for use according to any one of embodiments 1g to 7g wherein Compound (I), or a pharmaceutically acceptable salt thereof, is administered intravenously through infusion over a timeframe of about 40 minutes.
11g. Compound (I), or a pharmaceutically acceptable salt thereof, for use according to any one of embodiments 1g to 7g wherein Compound (I), or a pharmaceutically acceptable salt thereof, is administered intravenously in an amount of from about 0.0048 mg/kg to about 0.32 mg/kg, in particular 0.0048 mg/kg, 0.016 mg/kg, 0.048 mg/kg, 0.16 mg/kg and 0.32 mg/kg of Compound (I) per patient body weight.
12g. Compound (I), or a pharmaceutically acceptable salt thereof, for use according to embodiment 10a wherein Compound (I), or a pharmaceutically acceptable salt thereof, is administered once a week or once every other week.
13g. Compound (I), or a pharmaceutically acceptable salt thereof, for use according to any one of embodiments 1g to 5g wherein the use is in conjunction with one or more antidepressants and an antipsychotic or mood stabilizer.

### Embodiments (h)

1h. A pharmaceutical composition comprising Compound (I), or a pharmaceutically acceptable salt thereof, and at least one pharmaceutically acceptable excipient, for use in the treatment suicidality in a patient having major depressive disorder.
2h. A pharmaceutical composition comprising Compound (I), or a pharmaceutically acceptable salt thereof, and at least one pharmaceutically acceptable excipient, for use in the treatment of a patient having major depressive disorder with suicidal ideation.
3h. A pharmaceutical composition comprising Compound (I), or a pharmaceutically acceptable salt thereof, and at least one pharmaceutically acceptable excipient, for use in the treatment of a patient having major depressive disorder with suicidal ideation and suicidal intent.
4h. A pharmaceutical composition comprising Compound (I), or a pharmaceutically acceptable salt thereof, and at least one pharmaceutically acceptable excipient, for use in the treatment of a patient having major depressive disorder with suicidal behavior.
5h. A pharmaceutical composition comprising Compound (I), or a pharmaceutically acceptable salt thereof, and at least one pharmaceutically acceptable excipient, for use in the treatment of self-harm in a patient having major depressive disorder.
6h. A pharmaceutical composition comprising Compound (I), or a pharmaceutically acceptable salt thereof, and at least one pharmaceutically acceptable excipient, for use according to any one of embodiments 1 h to 5h wherein the use is in conjunction with one or more antidepressants.
7h. A pharmaceutical composition comprising Compound (I), or a pharmaceutically acceptable salt thereof, and at least one pharmaceutically acceptable excipient, for use according to any one of embodiments 1 h to 5h wherein the use is in conjunction with psychotherapy.
8h. A pharmaceutical composition comprising Compound (I), or a pharmaceutically acceptable salt thereof, and at least one pharmaceutically acceptable excipient, for use according to any one of embodiments 1 h to 5h wherein the use is in conjunction with one or more antidepressants and psychotherapy.
9h. A pharmaceutical composition comprising Compound (I), or a pharmaceutically acceptable salt thereof, and at least one pharmaceutically acceptable excipient, for use according to any one of embodiments 1 h to 8h wherein Compound (I), or a pharmaceutically acceptable salt thereof is administered intravenously.
10h. A pharmaceutical composition comprising Compound (I), or a pharmaceutically acceptable salt thereof, and at least one pharmaceutically acceptable excipient, for use according to any one of embodiments 1h to 8h wherein the pharmaceutical composition is administered intravenously through infusion over a timeframe of about 40 minutes.
11h. A pharmaceutical composition comprising Compound (I), or a pharmaceutically acceptable salt thereof, and at least one pharmaceutically acceptable excipient, for use according to any one of embodiments 1 h to 8h wherein the pharmaceutical composition is administered intravenously in an amount of from about 0.0048 mg/kg to about 0.32 mg/kg, in particular 0.0048 mg/kg, 0.016 mg/kg, 0.048 mg/kg, 0.16 mg/kg and 0.32 mg/kg of compound (I), or a pharmaceutically acceptable salt thereof per patient body weight.
12h. A pharmaceutical composition comprising Compound (I), or a pharmaceutically acceptable salt thereof, and at least one pharmaceutically acceptable excipient, for use according to embodiment 11h wherein the pharmaceutical composition is administered once a week or once every other week.
13h. A pharmaceutical composition comprising Compound (I), or a pharmaceutically acceptable salt thereof, and at least one pharmaceutically acceptable excipient, for use according to any one of embodiments 1 h to 5h wherein the use is in conjunction with one or more antidepressants and an antipsychotic or mood stabilizer.

### Embodiments (i)

2i. Use of Compound (I), or a pharmaceutically acceptable salt thereof, for the manufacture of a medicament for the treatment suicidality in a patient having major depressive disorder.
2i. Use of Compound (I), or a pharmaceutically acceptable salt thereof, for the manufacture of a medicament for the treatment of a patient having major depressive disorder with suicidal ideation.
3i. Use of Compound (I), or a pharmaceutically acceptable salt thereof, for the manufacture of a medicament for the treatment of a patient having major depressive disorder with suicidal ideation and suicidal intent.
4i. Use of Compound (I), or a pharmaceutically acceptable salt thereof, for the manufacture of a medicament for the treatment of a patient having major depressive disorder with suicidal behavior.
5i. Use of Compound (I), or a pharmaceutically acceptable salt thereof, for the manufacture of a medicament for the treatment of self-harm in a patient having major depressive disorder.
6i. Use of Compound (I), or a pharmaceutically acceptable salt thereof, according to any one of embodiments 1i to 5i wherein the use is in conjunction with one or more antidepressants.
7i. Use of Compound (I), or a pharmaceutically acceptable salt thereof, according to any one of embodiments 1i to 5i wherein the use is in conjunction with psychotherapy.
8i. Use of Compound (I), or a pharmaceutically acceptable salt thereof, according to any one of embodiments 1i to 5i wherein the use is in conjunction with one or more antidepressants and psychotherapy.
9i. Use of Compound (I), or a pharmaceutically acceptable salt thereof, according to any one of embodiments 1i to 8i wherein Compound (I), or a pharmaceutically acceptable salt thereof is administered intravenously.
10i. Use of Compound (I), or a pharmaceutically acceptable salt thereof, according to any one of embodiments 1i to 8i wherein Compound (I), or a pharmaceutically acceptable salt thereof is administered intravenously through infusion over a timeframe of about 40 minutes.
11i. Use of Compound (I), or a pharmaceutically acceptable salt thereof, according to any one of embodiments 1i to 8i wherein Compound (I), or a pharmaceutically acceptable salt thereof is administered intravenously in an amount of from about 0.0048 mg/kg to about 0.32 mg/kg, in particular 0.0048 mg/kg, 0.016 mg/kg, 0.048 mg/kg, 0.16 mg/kg and 0.32 mg/kg per patient body weight.
12i. Use of Compound (I), or a pharmaceutically acceptable salt thereof, according to embodiment 11c wherein Compound (I), or a pharmaceutically acceptable salt thereof is administered once a week or once every other week.
13i. Use of Compound (I), or a pharmaceutically acceptable salt thereof, according to any one of embodiments 1i to 5i wherein the use is in conjunction with one or more antidepressants and an antipsychotic or mood stabilizer.

### Embodiments (j)

1j. Use of a pharmaceutical composition comprising Compound (I), or a pharmaceutically acceptable salt thereof, and a pharmaceutically acceptable excipient, for the manufacture of a medicament for the treatment suicidality in a patient having major depressive disorder.
2j. Use of a pharmaceutical composition comprising Compound (I), or a pharmaceutically acceptable salt thereof, and a pharmaceutically acceptable excipient, for the manufacture of a medicament for the treatment of a patient having major depressive disorder with suicidal ideation.
3j. Use of a pharmaceutical composition comprising Compound (I), or a pharmaceutically acceptable salt thereof, and a pharmaceutically acceptable excipient, for the manufacture of a medicament for the treatment of a patient having major depressive disorder with suicidal ideation and suicidal intent.
4j. Use of a pharmaceutical composition comprising Compound (I), or a pharmaceutically acceptable salt thereof, and a pharmaceutically acceptable excipient, for the manufacture of a medicament for the treatment of a patient having major depressive disorder with suicidal behavior.
5j. Use of a pharmaceutical composition comprising Compound (I), or a pharmaceutically acceptable salt thereof, and a pharmaceutically acceptable excipient, for the manufacture of a medicament for the treatment of self-harm in a patient having major depressive disorder.
6j. Use of a pharmaceutical composition comprising Compound (I), or a pharmaceutically acceptable salt thereof, and a pharmaceutically acceptable excipient, according to any one of embodiments 1j to 5j wherein the use is in conjunction with one or more antidepressants.
7j. Use of a pharmaceutical composition comprising Compound (I), or a pharmaceutically acceptable salt thereof, and a pharmaceutically acceptable excipient, according to any one of embodiments 1j to 5j wherein the use is in conjunction with psychotherapy.
8j. Use of a pharmaceutical composition comprising Compound (I), or a pharmaceutically acceptable salt thereof, and a pharmaceutically acceptable excipient, according to any one of embodiments 1j to 5j wherein the use is in conjunction with one or more antidepressants and psychotherapy.
9j. Use of a pharmaceutical composition comprising Compound (I), or a pharmaceutically acceptable salt thereof, and a pharmaceutically acceptable excipient, according to any one of embodiments 1j to 8j wherein Compound (I), or a pharmaceutically acceptable salt thereof is administered intravenously.
10j. Use of a pharmaceutical composition comprising Compound (I), or a pharmaceutically acceptable salt thereof, and a pharmaceutically acceptable excipient, according to any one of embodiments 1j to 8j wherein Compound (I), or a pharmaceutically acceptable salt thereof is administered intravenously through infusion over a timeframe of about 40 minutes.
11j. Use of a pharmaceutical composition comprising Compound (I), or a pharmaceutically acceptable salt thereof, and a pharmaceutically acceptable excipient, according to any one of embodiments 1j to 8j wherein Compound (I), or a pharmaceutically acceptable salt thereof is administered intravenously in an amount of from about 0.0048 mg/kg to about 0.32 mg/kg, in particular 0.0048 mg/kg, 0.016 mg/kg, 0.048 mg/kg, 0.16 mg/kg and 0.32 mg/kg per patient body weight.
12j. Use of a pharmaceutical composition comprising Compound (I), or a pharmaceutically acceptable salt thereof, and a pharmaceutically acceptable excipient, according to embodiment 11j wherein Compound (I), or a pharmaceutically acceptable salt thereof is administered once a week or once every other week.
13j. Use of a pharmaceutical composition comprising Compound (I), or a pharmaceutically acceptable salt thereof, and a pharmaceutically acceptable excipient, according to any one of embodiments 1j to 5j wherein the use is in conjunction with one or more antidepressants and antipsychotic or mood stabilizer.

### Embodiments (k)

1k. A method for the treatment of suicidality in a patient having major depressive disorder comprising administration of an effective amount of Compound (I), or a pharmaceutically acceptable salt thereof, to a patient in need thereof.
2k. A method for the treatment of major depressive disorder with suicidal ideation comprising administration of an effective amount of Compound (I), or a pharmaceutically acceptable salt thereof, to a patient in need thereof.
3k. A method for the treatment of major depressive disorder with suicidal ideation and suicidal intent comprising administration of an effective amount of Compound (I), or a pharmaceutically acceptable salt thereof, to a patient in need thereof.
4k. A method for the treatment major depressive disorder with suicidal behavior comprising administration of an effective amount of Compound (I), or a pharmaceutically acceptable salt thereof, in a patient in need thereof.
5k. A method for the treatment of self-harm in a patient having major depressive disorder comprising administration of an effective amount of Compound (I), or a pharmaceutically acceptable salt thereof, to a patient in need thereof.
6k. The method according to any one of embodiments 1k to 5k wherein treatment is in conjunction with one or more antidepressants.
7k. The method according to any one of embodiments 1k to 5k wherein treatment is in conjunction with psychotherapy.
8k. The method according to any one of embodiments 1k to 5k wherein treatment is in conjunction with one or more antidepressants and psychotherapy.
9k. The method according to any one of embodiments 1k to 8k wherein Compound (I), or a pharmaceutically acceptable salt thereof is administered intravenously.
10k. The method according to any one of embodiments 1k to 8k wherein Compound (I), or a pharmaceutically acceptable salt thereof, is administered intravenously through infusion over a timeframe of about 40 minutes.
11k. The method according to any one of embodiments 1e to 8e wherein Compound (I), or a pharmaceutically acceptable salt thereof, is administered intravenously in an amount of from about 0.0048 mg/kg to about 0.32 mg/kg, in particular 0.0048 mg/kg, 0.016 mg/kg, 0.048 mg/kg, 0.16 mg/kg and 0.32 mg/kg per patient body weight.
12k. The method according to embodiment 11k wherein Compound (I), or a pharmaceutically acceptable salt thereof is administered once a week or once every other week.
13k. The method according to any one of embodiments 1e to 5e wherein treatment is in conjunction with one or more antidepressants and an antipsychotic or mood stabilizer.

### Embodiments (l)

1l. A method for the treatment of suicidality in a patient having major depressive disorder comprising administration of a pharmaceutical composition comprising an effective amount of Compound (I), or a pharmaceutically acceptable salt thereof, and a pharmaceutically acceptable excipient, to a patient in need thereof.
2l. A method for the treatment of major depressive disorder with suicidal ideation comprising administration a pharmaceutical composition comprising an effective amount of Compound (I), or a pharmaceutically acceptable salt thereof, and a pharmaceutically acceptable excipient, to a patient in need thereof.
3l. A method for the treatment of major depressive disorder with suicidal ideation and suicidal intent comprising administration of a pharmaceutical composition comprising an effective amount of Compound (I), or a pharmaceutically acceptable salt thereof, and a pharmaceutically acceptable excipient, to a patient in need thereof.
4l. A method for the treatment major depressive disorder with suicidal behavior comprising administration of a pharmaceutical composition comprising an effective amount of Compound (I), or a pharmaceutically acceptable salt thereof, and a pharmaceutically acceptable excipient, in a patient in need thereof.
5l. A method for the treatment of self-harm in a patient having major depressive disorder comprising administration of a pharmaceutical composition comprising an effective amount of Compound (I), or a pharmaceutically acceptable salt thereof, and a pharmaceutically acceptable excipient, to a patient in need thereof.
6l. The method according to any one of embodiments 1l to 5l wherein treatment is in conjunction with one or more antidepressants.
7l. The method according to any one of embodiments 1l to 5l wherein treatment is in conjunction with psychotherapy.
8l. The method according to any one of embodiments 1l to 5l wherein treatment is in conjunction with one or more antidepressants and psychotherapy.
9l. The method according to any one of embodiments 1l to 8l wherein the pharmaceutical composition is administered intravenously.
10l. The method according to any one of embodiments 1l to 8l wherein the pharmaceutical composition is administered intravenously through infusion over a timeframe of about 40 minutes.
11l. The method according to any one of embodiments 1l to 8l wherein the pharmaceutical composition is administered intravenously in an amount of from about 0.0048 mg/kg to about 0.32 mg/kg, in particular 0.0048 mg/kg, 0.016 mg/kg, 0.048 mg/kg, 0.16 mg/kg and 0.32 mg/kg. Compound (I), or a pharmaceutically acceptable salt thereof, per patient body weight.
12l. The method according to embodiment 11l wherein the pharmaceutical composition is administered once a week or once every other week.
13l. The method according to any one of embodiments 1l to 5l wherein treatment is in conjunction with one or more antidepressants and an antipsychotic or mood stabilizer.

The term “depressive symptoms”, as used herein, refers to symptoms associated with major depressive disorder and include one or more of the following:
Feeling sad or having a depressed mood
Loss of interest or pleasure in activities once enjoyed
Changes in appetite weight loss or gain unrelated to dieting
Trouble sleeping or sleeping too much
Loss of energy or increased fatigue
Increase in purposeless physical activity (e.g., inability to sit still, pacing, handwringing) or slowed movements or speech (these actions must be severe enough to be observable by others)
Feeling worthless or guilty
Difficulty thinking, concentrating or making decisions
Thoughts of death or suicide

The term “preparatory behavior”, as used herein, means acts or preparation toward making a suicide attempt, but before potential for harm has begun. This can include anything beyond a verbalization or thought, such as assembling a method (e.g., buying a gun, collecting pills) or preparing for one's death by suicide (e.g., writing a suicide note, giving things away).

The term “psychotherapy”, as used herein, refers to the informed and intentional application of clinical methods and interpersonal stances derived from established psychological principles for the purpose of assisting people to modify their behaviors, cognitions, emotions, and/or other personal characteristics in directions that the participants deem desirable.

The term “self-harm”, as used herein, means willful self-inflicting of painful, destructive, or injurious acts without intent to die.

The term “suicide”, as used herein means death caused by self-directed injurious behavior with any intent to die as a result of the behavior.

The term “suicidality”, as used herein, refers to thoughts of suicide (suicidal ideation) and suicide, suicide attempts and preparatory acts (suicidal behavior).

The term “suicide attempt”, as used herein, means a nonfatal self-directed potentially injurious behavior with any intent to die as a result of the behavior. A suicide attempt may or may not result in injury.

The term “suicidal behavior”, as used herein, includes suicide, suicide attempts, and acts to prepare for suicide.

The term “suicidal ideation”, as used herein, refers to passive thoughts about wanting to be dead or active thoughts about killing oneself, not accompanied by preparatory behavior. Suicidal ideation refers to thinking about or planning suicide. Thoughts can range from creating a detailed plan to having a fleeting consideration, but does not include the final act of suicide. Passive suicidal ideation is when there are thoughts of suicide or self-harm but no plan to carry it out. Active suicidal ideation is when there are thoughts of suicide or self-harm, and there is a plan to carry it out.

The terms “suicidal ideation with intent” or “suicidal Ideation with suicidal intent”, as used herein, refers to the passive thoughts about wanting to be dead or active thoughts about killing oneself with the desire, aim, purpose, or goal for a self-destructive act to end in death.

The term “suicidal ideation with no intent”, as used herein, refers to the passive thoughts about wanting to be dead or active thoughts about killing oneself, but with no intention to do so.

The term “treatment resistant depression (TRD)”, as used herein, refers to a type of major depressive disorder where the patient has not responded to adequate doses of two different antidepressants taken for a sufficient duration of time, which is usually six weeks.

The term “treat” “treating” “treatment” or “therapy”, as used herein, means obtaining beneficial or desired results, for example, clinical results. Beneficial or desired results can include, but are not limited to, alleviation of one or more symptoms of suicide, in particular suicide in patients with major depressive disorder, as defined herein, such as attempted suicide, suicidal ideation, suicidal intent, and suicidal behaviors. One aspect of the treatment is, for example, that said treatment should have a minimal adverse effect on the patient, e.g. the agent used should have a high level of safety, for example without producing the side effects of previously known treatment regimens. The term “alleviation”, for example in reference to a symptom of a condition, as used herein, refers to reducing at least one of the frequency and amplitude of a symptom of a condition in a patient.

As used herein, the term “subject” refers to a mammalian organism, preferably a human being (male or female).

As used herein, the term “patient” refers to a subject who is diseased and would benefit from the treatment.

As used herein, a subject is “in need of” a treatment if such subject (patient) would benefit biologically, medically or in quality of life from such treatment.

The term “pharmaceutical composition” is defined herein to refer to a mixture or solution containing at least one active ingredient or therapeutic agent to be administered to a subject, in order to treat a particular condition (i.e. disease, disorder or condition or at least one of the clinical symptoms thereof) affecting the subject.

As used herein, the term “pharmaceutically acceptable excipient” includes any and all solvents, dispersion media, coatings, surfactants, antioxidants, preservatives (e.g., antibacterial agents, antifungal agents), isotonic agents, absorption delaying agents, salts, preservatives, drug stabilizers, binders, excipients, disintegration agents, lubricants, sweetening agents, flavoring agents, dyes, and the like and combinations thereof, as would be known to those skilled in the art (see, for example, Remington's Pharmaceutical Sciences, 22^{nd} Ed. Mack Printing Company, 2013, pp. 1049-1070). Except insofar as any conventional carrier is incompatible with the active ingredient, its use in the therapeutic or pharmaceutical compositions is contemplated.

The terms “drug”, “active substance”, “active ingredient”, “pharmaceutically active ingredient”, “active agent” or “therapeutic agent” are to be understood as meaning a compound in free form or in the form of a pharmaceutically acceptable salt, in particular compounds of the type specified herein.

The term “intravenous or intravenously” refers to a pharmaceutical composition designed to be administered to a vein.

The term “mg/kg” as used herein refers to the amount of compound or drug, for example, Compound (I), or a pharmaceutically acceptable salt thereof, per kilogram of body weight of a patient or subject.

The term “pharmaceutical combination” refers to either a fixed combination in one unit dosage form (e.g., capsule, tablet, caplets or particulates), non-fixed combination, or a kit of parts for the combined administration where Compound (I) and one or more combination partner (e.g. another drug as specified herein, also referred to as further “pharmaceutically active ingredient”, “therapeutic agent” or “co-agent”) may be administered independently at the same time or separately within time intervals, especially where these time intervals allow that the combination partners show a cooperative, e.g. synergistic effect. The terms “co-administration” or “combined administration” or the like as utilized herein are meant to encompass administration of the selected combination partner to a single subject in need thereof (e.g. a patient), and are intended to include treatment regimens in which the agents are not necessarily administered by the same route of administration or at the same time. The term “fixed combination” means that the active ingredients, e.g. the compound of the present invention and one or more combination partners, are both administered to a patient simultaneously in the form of a single entity or dosage. The term “non-fixed combination” means that the active ingredients, e.g. a compound of the present invention and one or more combination partners, are both administered to a patient as separate entities either simultaneously or sequentially with no specific time limits, wherein such administration provides therapeutically effective levels of the two compounds in the body of the patient.

The compound of the present invention may be administered separately, by the same or different route of administration, or together in the same pharmaceutical composition as the other agents. In the combination therapies of the invention, the compound of the invention and the other therapeutic agent may be manufactured and/or formulated by the same or different manufacturers. Moreover, the compound of the invention and the other therapeutic may be brought together into a combination therapy: (i) prior to release of the combination product to physicians (e.g. in the case of a kit comprising the compound of the invention and the other therapeutic agent); (ii) by the physician themselves (or under the guidance of the physician) shortly before administration; (iii) in the patient themselves, e.g. during sequential administration of the compound of the invention and the other therapeutic agent.

As used herein, the term “a,” “an,” “the” and similar terms used in the context of the present invention (especially in the context of the claims) are to be construed to cover both the singular and plural unless otherwise indicated herein or clearly contradicted by the context.

The use of any and all examples, or exemplary language (e.g. “such as”) provided herein is intended merely to better illuminate the invention and does not pose a limitation on the scope of the invention otherwise claimed.

### EXPERIMENTAL

### Example 1. Clinical Evaluation Phase IIa

A multi-center, randomized, subject and investigator blinded, placebo controlled, active comparator, parallel group, proof of concept study to evaluate the efficacy, safety, tolerability, and pharmacokinetics of Compound (I) in patients with treatment resistant depression (TRD). The study included two different active Compound (I) doses of 0.16 mg/kg and 0.32 mg/kg given in two different regiments—weekly and bi-weekly (every other week). The study also included a ketamine arm.

Adults with major depressive disorder and prior failure of ≥2 standard antidepressants of adequate dose and >8 weeks duration in a major depressive episode (per DSM-5 criteria) and Montgomery-Åsberg Depression Rating Scale (MADRS) score ≥24 were eligible. Patients were randomized (3:3:3:3:6:4) to one of six treatment arms for 36 days: low dose (0.16 mg/kg) Compound (I) weekly (N=11) or every other week (q2w [N=10]); high dose (0.32 mg/kg) Compound (I) weekly (N=10) or q2w (N=9); placebo (N=20); or ketamine 0.5 mg/kg weekly (N=10). Change in MADRS total score at 24 hours (h) from baseline (primary endpoint), was analyzed using analysis of covariance. Secondary endpoints included change in MADRS score at 48 hours and 6 weeks and clinical global impression-improvement (CGI-I) score at 24 hours, 48 hours and 6 weeks. Safety and tolerability were also assessed.

At 24 hours, mean decreases in MADRS score were 15.51 points in the pooled Compound (I) 0.16 mg/kg group, 12.98 in the pooled Compound (I) 0.32 mg/kg group, 12.94 in the ketamine group and 7.27 in the placebo group. Adjusted mean differences (ΔAM) vs placebo at 24 hours were −8.25 (p=0.001), −5.71 (p=0.019) and −5.67 (p=0.046) and at 48 h were −7.06 (p=0.013), −7.37 (p=0.013), −11.02 (p=0.019) in the pooled Compound (I) 0.16 mg/kg, pooled Compound (I) mg/kg, and ketamine groups, respectively. While a greater improvement in MADRS score at Week 6 was observed in Compound (I) 0.32 mg/kg weekly (ΔAM [80% CI]: −5.42 [−10.8, −0.02], p=0.099) and ketamine groups (−5.24 [−10.4, −0.06], p=0.097) vs placebo, the greatest benefit was observed with Compound (I) 0.16 mg/kg q2w dose (−6.46 vs placebo [−11.8, −1.15], p=0.059). Results for Compound (I) vs placebo also suggested benefits for CGI-I scores at 24 hours, 48 hours and 6 weeks. Adverse events (AEs) occurred in 29%, 63%, 50% and 10% of patients in the pooled Compound (I) 0.16 mg/kg, Compound (I) 0.32 mg/kg, ketamine and placebo groups, respectively. The most common AEs in all treatment groups were amnesia, dissociation, sedation, and vomiting. Dissociative AEs occurred in 24%, 26%, 50% and 10% patients in the pooled Compound (I) 0.16 mg/kg, Compound (I) 0.32 mg/kg, ketamine and placebo groups, respectively and the corresponding incidence of sedation was 14%, 21%, 10% and 0%. Time to both onset and resolution of dissociative AEs and sedation was, on average, longer for Compound (I) than for ketamine.

In this proof-of-concept study, Compound (I) was associated with a significant improvement in MADRS score vs placebo in patients with TRD. No new safety signals were identified.

### Example 2. Clinical Evaluation Phase IIb

A double-blind, placebo-controlled, randomized dose-ranging study to investigate efficacy and safety of intravenous Compound (I) infusion in addition to pharmacological antidepressant treatment on the rapid reduction of symptoms of Major Depressive Disorder in subjects who have suicidal ideation with intent.

The main purpose of the study is to support the dose selection for future Phase 3 clinical trials by evaluating efficacy and safety of four Compound (I) doses (0.0048, 0.016, 0.048 and 0.16 mg/kg) administered every other week by intravenous infusion on top of pharmacological antidepressant treatment, compared with placebo for the rapid reduction of the symptoms of MDD in participants who have suicidal ideation with intent. In addition, the study will explore the effect of single dose administration of 0.16 and 0.048 mg/kg to treat MDD in participants who have suicidal ideation with intent.

The study will also have a 12-month Extension Period to explore durability of the effect of the study treatment and the potential of COMPOUND (I) to prevent relapses, as well as safety of repeated Compound (I) administration.

### Objectives and Endpoints

| Objective(s) | Endpoint(s) |
|---|---|
| Primary objective(s) | Endpoint(s) for primary objective(s) |
| Core and Extension: To | Change from baseline in MADRS total score at 24 |
| investigate dose response | hours after the start of the first infusion |
| relationship for 4 doses of | |
| COMPOUND (I) vs. placebo in | |
| change from baseline in | |
| MADRS total score at 24 hours | |
| after the start of the first | |
| intravenous infusion | |
| Secondary objective(s) | Endpoint(s) for secondary objective(s) |
| Core and Extension: | Number and severity of treatment-emergent adverse |
| To assess safety and | events (TEAEs), including AEs of special interest in the |
| tolerability of COMPOUND (I) | Core Period |
| Core and Extension: To | Proportion of participants meeting response criteria |
| assess the effect of | (≥50% reduction from baseline in MADRS total score) |
| COMPOUND (I) on sustained | at each scheduled visit/time point in the Core Period. |
| response and remission | Proportion of participants meeting criteria |
| | for sustained response (≥50% reduction from |
| | baseline in MADRS total score sustained for a period |
| | of at least four weeks) in the Core Period |
| | Proportion of participants meeting remission criteria |
| | (MADRS total score of ≤12) at each scheduled |
| | visit/time point in Core Period |
| | Proportion of participants meeting criteria for sustained |
| | remission (MADRS total score of ≤12 sustained for a |
| | period of at least four weeks) in the Core Period |
| | Proportion of participants meeting criteria for relapse |
| | over all randomized population over fixed period in |
| | Extension |
| | Proportion of relapsing participants meeting response |
| | criteria or remission criteria after the first infusion of |
| | COMPOUND (I) re-treatment in Extension Period |
| Core and Extension: To | PK properties of COMPOUND (I) in plasma after |
| assess COMPOUND (I) | 1^{st} infusion described by AUClast, Cmax, Tmax |
| pharmacokinetics in plasma | (parameters not limited) and after all following infusions |
| | described by Cmax and Tmax. |
| Exploratory objective(s) | Endpoint(s) for exploratory objective(s) |
| Core and Extension: To | Change from baseline in S-STS score at each |
| assess the effect of | scheduled post-baseline visit/ time point in Core Period |
| COMPOUND (I) on suicidality | Change from relapse in S-STS score at each |
| | scheduled post-baseline visit/ time point in Extension |
| | Period |
| | Proportion of participants meeting response criteria |
| | (≥50% improvement in S-STS total score) at each |
| | scheduled visit/ time post treatment in Core Period and |
| | Extension Period |
| | Proportion of participants meeting S-STS total score = |
| | 0 at each scheduled visit/ time post treatment in Core |
| | Period and Extension Period |
| | Proportion of participants meeting remission criteria |
| | (≥70% improvement in S-STS total score) at each |
| | scheduled visit/ time post treatment in Core Period and |
| | Extension Period |
| | Change from baseline in CGI-SS at 24 hours after the |
| | start of first infusion, and at each scheduled visit in |
| | Core Period |
| | Change from relapse in CGI-SS at each scheduled visit |
| | after re-treatment in Extension Period |
| | Proportion of participant achieving no suicidality |
| | evaluated by CGI-SS at each scheduled visit post |
| | treatment in Core Period and Extension Period |
| | Proportion of participants with suicidality improvement |
| | evaluated by CGIC-S at each scheduled visit post |
| | treatment in Core Period and Extension Period |
| Core and Extension: To | Change from baseline in CGI-S at 24 hours after the |
| assess the effect of | start of first infusion, and at each scheduled visit in |
| COMPOUND (I) on depressive | Core Period |
| mood | Change from relapse in CGI-S at each scheduled visit |
| | after re-treatment in Extension Period |
| | Proportion of participants achieving improvement |
| | evaluated by CGI-I after treatment in Core Period and |
| | after re-treatment in Extension Period |
| Core and Extension: To | Number of MDD related hospitalization days and MDD |
| assess the effect of | related re-hospitalization events in Core Period and |
| COMPOUND (I) on | Extension Period |
| hospitalization | |

### Primary Estimands

The primary clinical question of interest is: what is the effect of the Compound (I) versus placebo in change from baseline in MADRS total score at 24 hours post first dose administration, in conjunction with standard of care (SoC), in patients with MDD who have suicidal ideation with intent, accounting for intercurrent events (IEs) with potential confounding effects and IEs leading to study discontinuation prior to the 24 hours assessment.

This Phase 2b double-blind, placebo-controlled, randomized, parallel-group dose-ranging trial to investigate the efficacy and safety of four doses of intravenous Compound (I) 40-min infusion in addition to comprehensive standard of care (SoC) for the rapid reduction of the symptoms of MDD in participants who have suicidal ideation with intent. Comprehensive standard of care includes initial hospitalization and pharmacological antidepressant therapy (antidepressant monotherapy or an antidepressant plus augmentation). In addition, the study will also explore the effect for single dose administration of 0.16 mg/kg and 0.048 mg/kg given once to treat MDD in participants who have suicidal ideation with intent.

Participants meeting the eligibility criteria will be randomized in a 2:1:2:2:2:2:2 ratio to treatment with:
placebo every other week
Compound (I) 0.0048 mg/kg every other week
Compound (I) 0.016 mg/kg every other week
Compound (I) 0.048 mg/kg every other week
Compound (I) 0.16 mg/kg every other week
0.048 mg/kg single infusion with two subsequent placebo dosages given every other week
0.16 mg/kg single infusion with two subsequent placebo dosages given every other week on the top of ongoing pharmacological antidepressant SoC treatment. In all treatment arms, investigational treatment will be administered by intravenous infusion every other week, 3 infusions in total during the placebo-controlled double-blind Core, 6-week period of the study.

The study consists of three periods: a Screening period (up to 48 hrs), a Double-blind Core period (6 weeks) and Extension period (up to 12 months).

### Screening Period

The Screening period will start when the participant signs the informed consent form. The eligibility of the participant will be determined based on assessments performed at the screening visit (Day −2 to Day −1, up to −48 hrs) and also on Day 1 before randomization.

All participants must require hospitalization due to MDD with suicidal ideation with intent. Participants may have already been in the hospital prior to Screening or must agree to hospitalization for treatment in the study during Screening.

All participants must receive pharmacological antidepressant SoC, which either has been initiated prior to the study entry or must be initiated during the Screening period.

### Double-Blind Core Period

The Double-blind Core Period starts on Day 1. All baseline assessments (including the primary efficacy scale MADRS) must be performed on Day 1, prior to randomization. Investigational drug will be administered in a double-blind manner in the form of a 40-minute intravenous infusion on Day 1, Day 15 and Day 29. A designated unblinded staff member is required for the preparation of the infusion prior to administration.

### Hospitalization and Hospital Discharge.

The first intravenous infusion on Day 1 must be performed in an inpatient setting and the participant should remain hospitalized for a recommended observation period of 72 hours (with shorter or longer hospitalization duration allowed if clinically warranted per local standard practice), and as long as is required thereafter as per investigator's clinical judgment and local practice guidelines/recommendations. The minimum period of hospitalization is 24 hours after infusion on Day 1.

When hospitalization is no longer required as per investigator's clinical judgment and local practices, and the patient meets the minimum protocol discharge criteria (defined below), investigators should ensure that participants continue visiting the study site for investigational treatment (outpatient basis) and study assessments as per the visit schedule. During the second and third intravenous infusion (on Day 15 and Day 29), patients must be observed for at least 4 hours after the start of the infusion in an outpatient setting. Any relevant safety events (e.g. sedation, dissociation, amnesia, blood pressure increase or clinically relevant ECG findings) during the 4-hour observation period must be monitored at the site until resolution or absence of safety risk, as assessed by the investigator.

All patients who are ready for hospital discharge as per the investigator's clinical judgement must meet at least the following hospital discharge criteria as per below:
Participant does not have suicidal ideation with intent as confirmed by a “No” response to Question 3 AND Question 9 AND Question 10 obtained from the SSTS at the time of discharge. The participant should be free of suicidal ideation with intent for at least 24 hours before the discharge, as confirmed by the SSTS scale at the time of the assessment.
Absence of any safety risk assessed by the investigator based on clinical evaluation and judgement.

### Standard of Care/Psychotherapy

Dosage titration or adjustments of antidepressant pharmacological SoC treatment are allowed only during the first 2 weeks of double-blind treatment, as needed, with SoC dosages maintained thereafter during the Core period.

Psychotherapy is allowed if in a stable regimen at least 6 weeks before Screening and during the Core study treatment period.

### Double-Blind Extension Period

After completing the double-blind Core period all participants will be entering the Extension period for a minimum of 2 months.

Those participants who are not responders and not remitters at the end of the Core, will be observed for two months only to obtain safety data and evaluate withdrawal and rebound of the effect. Since those participants did not properly respond to the treatment in the first instance, no retreatment will be provided in this case. Those participants may change their standard of care treatment in their 2-month Extension period, if needed.

### Study Treatment

Participants who are classified as responders or remitters at the end of the Core period will be eligible for retreatment in case of a relapse in the Extension period, and should be observed for up to 12 months or up to the second relapse, whichever comes first. These participants will not be allowed to change their standard of care treatment until a relapse.

In the event of relapse participants will be administered with one course of the study treatment (3 IV infusions) in a dose-blinded manner. All participants will be pre-randomized to relapse retreatment already on Day 1 in the Core as described below:
Participants who were receiving Compound (I) in the Core Period will be assigned to the same dose and regimen for the treatment of relapse (if any in the Extension Period)
Participants who were randomized to placebo in the Core Period will be re-randomized in the Extension Period (1:2:2:2:2:2 ratio) to one of the active treatment arms in a dose-blinded manner for relapse treatment (if any in the Extension Period)
   If, based on the Core period study results, an optimal dose and dose regimen are established prior to the participant receiving re-treatment in Extension period, the participant originally randomized to a non-optimal Compound (I) dose and/or regimen will receive the optimal dose and regimen in a dose-blinded manner for the treatment of relapse. Participants will be assigned to the optimal dose and regimen through IRT in a blinded manner.

### Standard of Care/Psychotherapy

During the Extension period, pharmacological antidepressant therapy can be modified only for those participants who are not eligible for retreatment (not responders or who did not achieve remission). Those participants, who are classified as a responder or remitter at the end of the Core period should be stable on the SoC and as far as feasible stable on psychotherapy until the time of a relapse (if any) in the Extension period. In case of a relapse, the SoC pharmacological treatment can be adjusted, if needed, during the first 2 weeks of relapse treatment. Participants may or may not be hospitalized for the treatment of relapse (as per clinical judgment and local practices). Similarly, if at the End of relapse retreatment visit the patient is classified as a responder or remitter, similar approach should be followed with regards to the standard of care treatment. In case of a second relapse, the patient should be discontinued from the trial.

### Extension Period Duration

The end of the study/Extension period for participants (study completer) will be declared when one of the following is met:
All participants must complete the first 2-months of the Extension (Follow-up).
For participants who at the end of Core Period (Day 43, Visit 170) did not achieve remission and are not responders, these study participants should complete 2 months of follow-up.
For participants who are classified as responders or remitters at the end of the Core Period (Day 43, Visit 170), and who do not experience any relapse, the Extension Period will last 12 months (52 weeks).
For participants who are classified as responders or remitters at the end of the Core Period (Day 43, Visit 170), and relapse within 12 months (52 weeks) of the Extension period, they will receive one course of re-treatment of Compound (I) (as described above). If, after the retreatment they are classified as responders or remitters again as per (scores should be compared to the MADRS score at the time of relapse confirmation), they should continue the Extension period for up to 12 months or time to the second relapse, whichever comes first. A minimum 2 months follow-up observation must be conducted for these patients. If, at the end of Relapse Retreatment Visit the patient is not classified as a responder or remitter, this patient should be observed for 2 months after the end of their relapse treatment and discontinued the Extension thereafter.

### Study Population

The study population will consist of male and female participants 18 years old to 65 years old with MDD who have suicidal ideation with intent. The goal is to randomize a total of approximately 195 participants in approximately 50-60 centers worldwide. Randomized patients that prematurely discontinue will not be replaced.

### Inclusion Criteria

Participants eligible for inclusion in this study must meet all of the following criteria:
1. Signed informed consent must be obtained prior to participation in the study
2. Male and female participants, 18 to 65 years of age (inclusive) at screening
3. DSM-5 defined major depressive episode (MDE) without psychotic features at the time of screening based upon clinical assessment and confirmed by the Mini International Psychiatric Interview (MINI)
4. Participants must have current suicidal ideation with intent, confirmed by a “Yes” response to Question B3 AND either Question B10 or Question B11 obtained from the MINI Suicidality
5. Current suicidal ideation with intent, confirmed by “Yes” response to Question 3 AND either Question 9 or Question 10 obtained from the SSTS at Baseline
6. Persistent suicidal ideation with intent, confirmed by a “Yes” response to SSTS additional eligibility question at Baseline [“Did you spend at least 15 minutes per day having suicidal thoughts, impulses or behavior(s), most days for the 7 days prior to today's baseline visit (4/7) and at least one day after screening? YES/N0”].
7. Montgomery-Åsberg Depression Rating Scale (MADRS) score >28 at Screening and before randomization on Day 1
8. Participants must agree to receive pharmacological standard of care treatment to treat their MDD (as determined by the treating physician(s) based on clinical judgement and local treatment guidelines) during the trial duration
9. In the physician's opinion, acute psychiatric hospitalization is clinically warranted to treat the patient's condition, and the patient is already in the hospital or agrees to be hospitalized voluntarily for the required per protocol period.

### Exclusion Criteria

Participants meeting any of the following criteria are not eligible for inclusion in this study.
1. Any prior or current diagnosis of bipolar disorder, MDD with psychotic features, schizophrenia, or schizoaffective disorder at screening
2. Patients with acute alcohol or substance use disorder or withdrawal symptoms requiring detoxification, or patients who went through detoxification treatment (inpatient or outpatient) within 1 month before Screening
3. Participant has a current clinical diagnosis of autism, dementia, or intellectual disability
4. History of seizures. Note: a single childhood febrile seizure is not exclusionary
5. Participants with borderline personality disorder
6. Participants with suicidal ideation or behavior caused primarily by another non-MDD condition, as obtained from MINI Suicidality Disorders Classification Interview
7. Prior suicidality due to a lack of effect with ketamine or esketamine
8. Active hepatitis B virus (HBV), hepatitis C virus (HCV), human immunodeficiency virus (HIV) or active COVID infection as per medical history and/or available medical records
9. History of hypersensitivity to any of the study drugs or its excipients or to drugs of similar chemical classes.
10. Participants taking medications prohibited by the protocol.
11. Intake of the following medications/psychotherapy:
   Esketamine or ketamine 2 months before Screening
   Monoamine oxidase inhibitors (MAOIs) 14 days before Screening
   Non-stable psychotherapy regimen and/or started less than 6 weeks before Screening
12. Cardiac or cardiac repolarization abnormality, including any of the following:
   History of myocardial infarction (MI), angina pectoris, or coronary artery bypass graft (CABG) within 6 months prior to starting study treatment
   Clinically significant cardiac arrhythmias (e.g., ventricular tachycardia), complete left bundle branch block, high-grade AV block (e.g., bifascicular block, Mobitz type II and third degree AV block)
13. Resting QTcF 450 msec (male) or ≥460 msec (female) at Screening or pre first dose on Day 1, or inability to determine the QTcF interval
14. Risk factors for Torsades de Pointes (TdP) including uncorrected hypokalemia or hypomagnesemia, history of cardiac failure, or history of clinically significant/symptomatic bradycardia or any of the following:
   Long QT syndrome, family history of idiopathic sudden death or congenital long QT syndrome
   Concomitant medication(s) with a “Known Risk of Torsades de Pointes” that cannot be discontinued or replaced by safe alternative medication 7 days prior to Screening and during the Core.
15. Participant has uncontrolled hypertension (systolic blood pressure >140 mmHg or diastolic blood pressure >90 mmHg) despite diet, exercise or a stable dose of an allowed anti-hypertensive treatment at Screening and pre first dose on Day 1; or any past history of hypertensive crisis
16. Any other condition (e.g. known liver disease/liver dysfunction, active malignancy, etc.) which in the opinion of the investigator would put the safety of the participant at risk, impede compliance or hinder completion of the study.
17. Have evidence of significant renal insufficiency, indicated by an estimated glomerular filtration rate (eGFR) of <40 mL/min/1.73 m² at Screening
18. Use of other investigational drugs within 30 days of Screening
19. Pregnant or nursing (lactating) women
20. Sexually active males unwilling to use a condom during intercourse while taking study treatment and for 1 week after stopping study treatment (in the Core and Extension in case of retreatment). A condom is required for all sexually active male participants to prevent them from fathering a child AND to prevent delivery of study treatment via seminal fluid to their partner. In addition, male participants must not donate sperm for the period specified above.
21. Women of child-bearing potential, defined as all women physiologically capable of becoming pregnant, unless they are using highly effective methods of contraception while taking study treatment and for 1 week after stopping medication (applies to the Core and Extension). Highly effective contraception methods include:
   Total abstinence (when this is in line with the preferred and usual lifestyle of the participant). Periodic abstinence (e.g., calendar, ovulation, symptothermal, post-ovulation methods) and withdrawal are not acceptable methods of contraception
   Female sterilization (have had surgical bilateral oophorectomy with or without hysterectomy) total hysterectomy or tubal ligation at least six weeks before taking investigational drug. In case of oophorectomy alone, only when the reproductive status of the woman has been confirmed by follow up hormone level assessment
   Male sterilization (at least 6 months prior to screening). For female participants on the study, the vasectomized male partner should be the sole partner for that participant
   Use of an intrauterine device (IUD) or intrauterine system (IUS).
Oral contraception or systemic hormonal contraception (e.g. transdermal or implanted hormonal methods) is not allowed for the purpose of contraception.
Women are considered post-menopausal and not of child bearing potential if they have had 12 months of natural (spontaneous) amenorrhea with an appropriate clinical profile (e.g. age appropriate, history of vasomotor symptoms) or have had surgical bilateral oophorectomy (with or without hysterectomy), total hysterectomy or tubal ligation at least six weeks ago. In the case of oophorectomy alone, only when the reproductive status of the woman has been confirmed by follow up hormone level assessment is she considered not of child bearing potential. Patients eligible to receive Compound (I) treatment for relapse in the Extension Period should not be pregnant prior to the start of the treatment.

### Response and Remission Criteria

Response is defined as a 50% reduction from the baseline MADRS score at any time of the study. Remission is defined as MADRS total score of points at any time of the study.

Participants who completed the Core period on study treatment and meet one of the above response or remission criteria at the end of the Core period (Day 43, Visit 170) will be eligible for relapse retreatment in the Extension Period.

Worsening of depressive or suicidal symptoms during the Core is not considered a relapse and, if clinically significant, should be reported as an AE in the Core period (e.g. worsening of depression, increased suicidal thoughts, etc).

### Relapse Criteria (Extension Period)

Participants who completed the Core period on study treatment and meet one of the above response or remission criteria at the end of the Core period (Day 43, Visit 170) will be eligible for relapse retreatment in the Extension Period.

A relapse manifests as appearance of new depressive symptoms or worsening of previously stable or improving MDD symptoms. During the Extension Period, participants experiencing deterioration must be assessed by the treating physician and the relapse must be confirmed by assessment of the MADRS. Meeting any of the following criteria will be considered a relapse:
All participants independently if they achieved remission (MADRS≤12) or ≥50% improvement at the end of Core, must have MADRS total score ≥22 for 2 consecutive visits separated by 5-14 days and meet DSM-5 criteria for MDD to be eligible for relapse retreatment. In addition, those participants who demonstrated treatment response at the end of Core period (≥50% improvement) but did not achieve remission (MADRS≤12), should demonstrate ≥50% worsening from their MADRS total score reported at the end of Core Period (End of Core Visit).
   Two consecutive assessments separated by 5-14 days should confirm the relapse and the date of the first MADRS assessment will be the date of relapse. The relapse confirmation can be performed either at the Pre-retreatment Visit or before the first infusion given for retreatment. For the confirmation of the second relapse, it can be done at any time point after the course of the retreatment (two MADRS assessments separated by 5-14 days are still required).

### OR

Hospitalization for worsening depression or any other clinically relevant event suggestive of a relapse (i.e. suicide attempt, or hospitalization for suicide prevention). Such patients should meet the criteria of DSM-5 criteria for MDD to be considered in the relapse state (independently of the MADRS score). If a participant is hospitalized for any of these events, the start date of hospitalization will be used for the date of relapse. Otherwise the date of the event will be used if the participant is not hospitalized.
   In case a number of relapse criteria are met, the earlier date will be defined as the start date of relapse for this participant.

### Assessments:

### The Montgomery Asberg Depression Rating Scale (MADRS), SIGMA Version

The Montgomery Åsberg Depression Rating Scale (MADRS, SIGMA version), is a clinician-rated scale designed to measure depression severity and detects changes due to antidepressant treatment. The test consists of 10 items, each of which is scored from 0 (item not present or normal) to 6 (severe or continuous presence of the symptoms), for a total possible score of 60. Higher scores represent a more severe condition. The MADRS evaluates apparent sadness, reported sadness, inner tension, sleep, appetite, concentration, lassitude, interest level, pessimistic thoughts and suicidal thoughts (Khan et al 2000). The test exhibits high inter-rater reliability.

The structured interview guide for the MADRS (SIGMA) will be used for each administration. The MADRS will be administered electronically by qualified personnel.

In this study, 2 recall periods will be used: “Last 7 days” and “Since last visit/evaluation”. For the 4 hours timepoint, the sleep and appetite items will not be assessed (predose scores for these items obtained on the same day will be carried forward).

### Clinical Outcome Assessments (COAs)

To assess suicidality in participants, the following assessments are to be conducted:
Sheehan Suicidality Tracking Scale (S-STS), clinician-administered version
Clinical Global Impression of Severity of Suicidality Scale (CGI-SS)
Clinical Global Impression of Change in Suicidality scale (CGIC-S)
Clinical Global Impressions Scale-Severity (CGI-S)
Clinical Global Impressions Scale-Improvement (CGI-I)
   All secondary suicidality scales are psychometrically validated and reliable as measures of subtypes or symptom features of depressive states. They are being used to explore possible subtype effects or associated symptom changes to clarify which aspects of depression may improve more or less with the interventions given.

### Sheehan Suicidality Tracking Scale (S-STS) Standard Version, Clinician Rated

The Sheehan-Suicidality Tracking Scale (S-STS) is a scale designed to assess and monitor suicidality over time. The standard, clinician-rated version of the scale will be used in this trial. The standard version of the scale was designed for use in clinical research studies as a safety assessment measure, to detect treatment emergent suicidality, and as a treatment outcome measure that is very sensitive to change. The standard version of the S-STS is a 16-item scale that assesses the seriousness of suicidality phenomena on a Likert-type scale (0-4) ranging from “not at all” (0) to “extremely” (4). It also assesses the frequency of key phenomena and the overall time spent in suicidality Sheehan et al 2014.

The S-STS will be administered electronically by qualified site personnel. Different recall periods will be used in the study: “Last 24 hours”, “Last 7 days”, “Since last visit/evaluation”

### Clinical Global Impression of Severity of Suicidality Scale (CGI-SS)

The Clinical Global Impression of Severity of Suicidality (CGI-SS) is a 5 point scale that measures the patient's level of suicidality over time. Different recall periods will be used in the study: “Last 24 hours”, “Last 7 days”, “Since last visit/evaluation”. The CGI-SS will be assessed electronically by qualified personnel.

### Clinical Global Impression of Change in Suicidality Scale (CGIC-S)

The Clinical Global Impression of Change in Suicidality (CGIC-S) is a 7-point scale, providing an assessment by the investigator of the participant's suicidality compared to baseline. The CGIS-S will be administered electronically by qualified personnel.

### Clinical Global Impressions Scale-Severity (CGI-S) for Depressive Mood

The Clinical Global Impression (CGI) is an observer-rated scale that measures the severity of symptoms, treatment response and the efficacy of treatments in patients with mental disorders. The CGI-Severity (CGI-S) an instrument designed to assess the overall degree of illness at any point in time. The CGI-Severity (CGI-S) for depressive mood will be used in this trial.

The CGI-S assessment will be administered electronically by qualified personnel. Different recall periods will be used in the study: “Last 24 hours”, “Last 7 days”, “Since last visit/evaluation”

### Clinical Global Impressions Scale-Improvement (CGI-I) for Depressive Mood

The Clinical Global Impression (CGI) is a observer-rated scale which measures the severity of symptoms, treatment response and the efficacy of treatments in patients with mental disorders. The CGI-Improvement (CGI-I) (Guy, 1976) is assessing the degree of illness change (improvement or worsening) relative to a baseline.

The CGI-I will be administered electronically by qualified personnel.

1. A method for the treatment of major depressive disorder comprising administering an effective amount of Compound (I), or a pharmaceutically acceptable salt thereof, 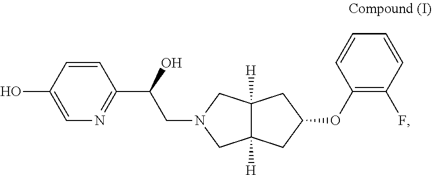 [SMILES: Oc1ccc([C@@H](O)CN2C[C@H]3C[C@H](Oc4ccccc4F)C[C@H]3C2)nc1]
to a patient in need thereof.

2. A method for the treatment of treatment resistant depression comprising administering an effective amount of Compound (I), or a pharmaceutically acceptable salt thereof, 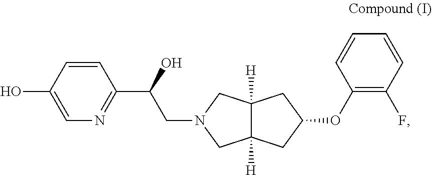 [SMILES: Oc1ccc([C@@H](O)CN2C[C@H]3C[C@H](Oc4ccccc4F)C[C@H]3C2)nc1]
to a patient in need thereof.

3-4. (canceled)

5. A method for the treatment of major depressive disorder (MDD) with suicidal ideation comprising administering an effective amount of Compound (I), or a pharmaceutically acceptable salt thereof, 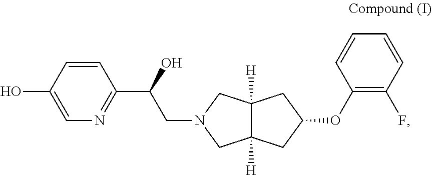 [SMILES: Oc1ccc([C@@H](O)CN2C[C@H]3C[C@H](Oc4ccccc4F)C[C@H]3C2)nc1]
to a patient in need thereof.

6. A method for the treatment of major depressive disorder (MDD) with suicidal ideation with suicidal intent comprising administering an effective amount of Compound (I), or a pharmaceutically acceptable salt thereof, 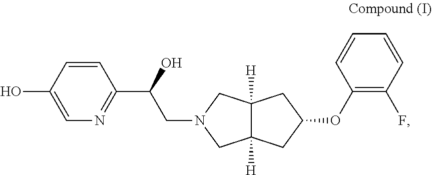 [SMILES: Oc1ccc([C@@H](O)CN2C[C@H]3C[C@H](Oc4ccccc4F)C[C@H]3C2)nc1]
to a patient in need thereof.

7-8. (canceled)

9. The method according to claim 1, wherein treatment is in conjunction with one or more antidepressants other than Compound (I), or the pharmaceutically acceptable salt thereof.

10. The method according to claim 1, wherein treatment is in conjunction with psychotherapy.

11. (canceled)

12. The method according to claim 1, wherein Compound (I), or the pharmaceutically acceptable salt thereof, is administered intravenously.

13. (canceled)

14. The method according to claim 1, wherein Compound (I), or the pharmaceutically acceptable salt thereof, is administered twice a week, once a week, once every other week, once a month, or once every six weeks.

15. The method according to claim 14 wherein Compound (I), or the pharmaceutically acceptable salt thereof, is administered once a week, once every other week, or once a month in an amount of about 0.0048 mg/kg, 0.016, 0.048 mg/kg, 0.16 mg/kg, or 0.32 mg/kg.

16. (canceled)

17. The method according to claim 1, wherein treatment is in conjunction with one or more antidepressants other than Compound (I), or the pharmaceutically acceptable salt thereof, and an antipsychotic or a mood stabilizer.

18-20. (canceled)

21. The method according to claim 2, wherein treatment is in conjunction with one or more antidepressants other than Compound (I), or the pharmaceutically acceptable salt thereof.

22. The method according to claim 2, wherein treatment is in conjunction with psychotherapy.

23. The method according to claim 2, wherein Compound (I), or the pharmaceutically acceptable salt thereof, is administered intravenously.

24. The method according to claim 2, wherein Compound (I), or the pharmaceutically acceptable salt thereof, is administered twice a week, once a week, once every other week, once a month, or once every six weeks.

25. The method according to claim 24, wherein Compound (I), or the pharmaceutically acceptable salt thereof, is administered once a week, once every other week, or once a month in an amount of about 0.0048 mg/kg, 0.016 mg/kg, 0.048 mg/kg, 0.16 mg/kg, or 0.32 mg/kg.

26. The method according to claim 2, wherein treatment is in conjunction with one or more antidepressants other than Compound (I), or the pharmaceutically acceptable salt thereof, and an antipsychotic or a mood stabilizer.

27. The method according to claim 5, wherein treatment is in conjunction with one or more antidepressants other than Compound (I), or the pharmaceutically acceptable salt thereof.

28. The method according to claim 5, wherein treatment is in conjunction with psychotherapy.

29. The method according to claim 5, wherein Compound (I), or the pharmaceutically acceptable salt thereof, is administered intravenously.

30. The method according to claim 5, wherein Compound (I), or the pharmaceutically acceptable salt thereof, is administered twice a week, once a week, once every other week, once a month, or once every six weeks.

31. The method according to claim 30, wherein Compound (I), or the pharmaceutically acceptable salt thereof, is administered once a week, once every other week, or once a month in an amount of about 0.0048 mg/kg, 0.016 mg/kg, 0.048 mg/kg, 0.16 mg/kg, or 0.32 mg/kg.

32. The method according to claim 5, wherein treatment is in conjunction with one or more antidepressants other than Compound (I), or the pharmaceutically acceptable salt thereof, and an antipsychotic or a mood stabilizer.

33. The method according to claim 6, wherein treatment is in conjunction with one or more antidepressants other than Compound (I), or the pharmaceutically acceptable salt thereof.

34. The method according to claim 6, wherein treatment is in conjunction with psychotherapy.

35. The method according to claim 6, wherein Compound (I), or the pharmaceutically acceptable salt thereof, is administered intravenously.

36. The method according to claim 6, wherein (Compound (I), or the pharmaceutically acceptable salt thereof, is administered twice a week, once a week, once every other week, once a month, or once every six weeks.

37. The method according to claim 36, wherein Compound (I), or the pharmaceutically acceptable salt thereof, is administered once a week, once every other week, or once a month in an amount of about 0.0048 mg/kg, 0.016 mg/kg, 0.048 mg/kg, 0.16 mg/kg, or 0.32 mg/kg.

38. The method according to claim 6, wherein treatment is in conjunction with one or more antidepressants other than Compound (I), or the pharmaceutically acceptable salt thereof, and an antipsychotic or a mood stabilizer.
